(19) United States
(12) Patent Application Publication
(10) Pub. No.: US 2026/0041553 A1
(43) Pub. Date: Feb. 12, 2026

(54) OUTER SKIRTS FOR PROSTHETIC VALVES

(71) Applicant: EDWARDS LIFESCIENCES CORPORATION, Irvine, CA (US)

(72) Inventors: Sandip Vasant Pawar, Irvine, CA (US); Radhika Vaid, Rancho Mission Viejo, CA (US); Michael Bukin, Pardes Hanna (IL); Gillian Irene Armstrong, Long Beach, CA (US); Nikolai Gurovich, Hadera (IL); Giolnara Pinhas, Hadera (IL)

(73) Assignee: EDWARDS LIFESCIENCES CORPORATION, Irvine, CA (US)

(21) Appl. No.: 19/364,155

(22) Filed: Oct. 21, 2025

Related U.S. Application Data
(63) Continuation of application No. PCT/US2024/025659, filed on Apr. 22, 2024.
(60) Provisional application No. 63/462,001, filed on Apr. 26, 2023.

(51) Int. Cl. A61F 2/24

(52) U.S. Cl. CPC A61F 2/2418; A61F 2/2415; A61F 2220/0075; A61F 2240/001

(57) ABSTRACT

Disclosed herein are prosthetic valves with outer skirts that include patterned threads extending over base layers thereof to provide PVL sealing functionality, and methods for forming such patterned outer skirts. As one example, a prosthetic valve can include an annular frame and an outer skirt disposed around the frame, wherein the outer skirt comprises a base layer, a multi-yarn thread comprising a plurality of multi-filament yarns, and a backing thread. The multi-yarn thread can be embroidered to the base layer and define outer portions extending over an outer surface of the base layer, while the backing layer extends over an inner surface of the base layer, and is wound over the multi-yarn thread at stitch points defined at ends of the outer portions.

### CROSS-REFERENCE TO RELATED APPLICATIONS

This application is a continuation of International Application No. PCT/US2024/025659, filed Apr. 22, 2024, which claims the benefit of U.S. Provisional Application No. 63/462,001, filed Apr. 26, 2023, which is incorporated by reference herein.

### FIELD

The present invention relates to the field of implantable prosthetic heart valves, and more particularly, to outer skirts having specific patterns and suture characteristics, methods of preparation thereof and implantable prosthetic heart valves comprising the same.

### BACKGROUND

Native heart valves, such as the aortic, pulmonary and mitral valves, function to assure adequate directional flow from, and to, the heart, and between the heart's chambers, to supply blood to the whole cardiovascular system. Various valvular diseases can render the valves ineffective and require replacement with artificial valves. Surgical procedures can be performed to repair or replace a heart valve. Since surgeries are prone to an abundance of clinical complications, alternative less invasive techniques of delivering a prosthetic heart valve over a catheter and implanting it over the native malfunctioning valve have been developed over the years.

Different types of prosthetic heart valves are known to date, including balloon expandable valve, self-expandable valves and mechanically-expandable valves. Different methods of delivery and implantation are also known, and may vary according to the site of implantation and the type of prosthetic valve. One exemplary technique includes utilization of a delivery assembly for delivering a prosthetic valve in a crimped state, from an incision which can be located at the patient's femoral or iliac artery, toward the native malfunctioning valve. Once the prosthetic valve is properly positioned at the desired site of implantation, it can be expanded against the surrounding anatomy, such as an annulus of a native valve, and the delivery assembly can be retrieved thereafter.

### SUMMARY

Most expandable, prosthetic valves comprise an annular frame and prosthetic leaflets mounted inside the frame. These valves can also include one or more skirts spanning a circumference of the frame, on an inner or outer surface of the frame. These skirts can be configured to establish a seal with the surrounding native tissue when the prosthetic valve is placed at the implantation site, so as to seal thereagainst. However, the native tissue (e.g., at the native valve annulus or arterial wall around the native valve) can have an irregular shape while the frame of the prosthetic valve is generally cylindrical. As a result, gaps can be formed between the prosthetic valve and native heart valve when the prosthetic valve is implanted within the native heart valve, even when skirts are disposed around the periphery of the prosthetic valve. Accordingly, a need exists for improved skirts for prosthetic valves which can better fill gaps between the native tissue and the prosthetic valve.

In one of its basic configurations, a prosthetic valve comprises an outer skirt comprising a multi-yarn thread. This basic configuration can preferably be provided with any one or more of the features described elsewhere herein, in particular with those of the examples described hereafter. However, it should be understood that the basic configuration can preferably also be provided with any one or more of the features shown in the figures and/or described in conjunction with the figures, either in addition to or alternatively to the features of the examples described hereafter.

In some examples, the prosthetic valve can comprise an annular frame movable between a radially compressed state and a radially expanded state.

In some examples, the outer skirt is optionally disposed around the frame.

In some examples, the outer skirt can comprise a base layer having an inner surface facing the frame and an outer surface facing away from the frame.

In some examples, the multi-yarn thread can comprise a plurality of multi-filament yarns.

In some examples, the multi-yarn thread can define a first thickness in a free state thereof.

In some examples, the multi-yarn thread can comprise a backing thread defining a second thickness.

In some examples, the multi-yarn thread optionally define at least one outer portion extending over the outer surface of the base layer, between two stitch points extending through the base layer.

In some examples, the multi-yarn thread is optionally wound around the backing thread at the two stitch points.

In some examples, the backing thread optionally extends along the inner surface of the base layer, in parallel to the corresponding outer portion, between the corresponding two stitch points.

In some examples, the first thickness is optionally greater than the second thickness.

In some examples, the multi-yarn thread can comprise between 4 and 16 multi-filament yarns.

In some examples, the multi-yarn thread is optionally coupled to the base layer at the two stitch points.

In some examples, each of the yarns of the multi-yarn thread can optionally have a weight between 20 Denier to 80 Denier.

In some examples, the multi-yarn thread can optionally have a filament count between 10 to 48 per yarn.

In one of its basic methods, an embroidery patterning method for forming and assembling an outer skirt of a prosthetic valve comprises providing an automated sewing machine, which comprises an embroidery needle and an embroidery bobbin assembled to a rotary hook. This basic method can preferably be provided with any one or more of the steps described elsewhere herein, in particular with those of the examples described hereafter. However, it should be understood that the basic method can preferably also be provided with any one or more of the steps shown in the figures and/or described in conjunction with the figures, either in addition to or alternatively to the steps of the examples described hereafter.

In some examples, the method comprises providing a backing thread.

In some examples, the backing thread is optionally coiled around the embroidery bobbin.

In some examples, the method comprises providing a multi-yarn thread.

In some examples, the multi-yarn thread is optionally held by the embroidery needle.

In some examples, the method comprises spreading a base layer in the automated sewing machine such that its inner surface is facing the embroidery bobbin and its outer surface is facing the embroidery needle.

In some examples, the method comprises pulling a section of the backing thread from the embroidery bobbin toward the inner surface of the base layer at a first stitch point.

In some examples, the method comprises passing the needle with the multi-yarn thread through the base layer at the first stitch point.

In some examples, the method comprises lacing the multi-yarn thread around the backing thread, optionally using the rotary hook.

In some examples, the method can comprise withdrawing the embroidery needle from the base layer, thereby forming a stitch of the multi-yarn thread and the backing thread at the first stitch point.

In some examples, the method can comprise moving the base layer to position a subsequent stitch point between the embroidery needle and the embroidery bobbin.

In some examples, the method can comprise repeating the steps of pulling the section of the backing thread, passing the needle with the multi-yarn thread, lacing the multi-yarn thread, and withdrawing the embroidery needle, to form an additional stitch at the subsequent stitch point, thereby forming an outer portion of the multi-yarn thread optionally extending between the first stitch point and the subsequent stitch point.

In some examples, the method can comprise attaching the base layer to a frame of a prosthetic valve, optionally such that the inner surface of the base layer is facing the frame.

In one of its basic configurations, a prosthetic valve comprises an annular frame and an outer skirt comprising one or more threads that define a plurality of outer portions. This basic configuration can preferably be provided with any one or more of the features described elsewhere herein, in particular with those of the examples described hereafter. However, it should be understood that the basic configuration can preferably also be provided with any one or more of the features shown in the figures and/or described in conjunction with the figures, either in addition to or alternatively to the features of the examples described hereafter.

In some examples, the annular frame defines a longitudinal axis and is optionally movable between a radially compressed state and a radially expanded state.

In some examples, the outer skirt is optionally disposed around the frame.

In some examples, the outer skirt can comprise a base layer extending between an inflow edge portion and an outflow edge portion.

In some examples, the base layer optionally has an inner surface facing the frame and an outer surface facing away from the frame.

In some examples, each outer portion optionally extends over the outer surface of the base layer, between two stitch points of a plurality of stitch points at which the thread is coupled to the base layer.

In some examples, the plurality of stitch points can comprise a plurality of inflow stitch points closer to the inflow edge portion relative to the rest of the plurality of stitch points.

In some examples, the plurality of stitch points can comprise a plurality of outflow stitch points closer to the outflow edge portion relative to the rest of the plurality of stitch points.

In some examples, the plurality of stitch points can comprise a plurality of first intermediate stitch points proximal to the inflow stitch points.

In some examples, the plurality of stitch points can comprise a plurality of second intermediate stitch points distal to the outflow stitch points.

In some examples, the plurality of outer portions can comprise a plurality of first diagonal portions.

In some examples, each first diagonal portion optionally extends between one of the inflow stitch points and one of the outflow stitch points.

In some examples, the plurality of outer portions can comprise a plurality of second diagonal portions.

In some examples, each second diagonal portion can extend between one of the inflow stitch points and one of the first intermediate stitch points.

In some examples, the plurality of outer portions can comprise a plurality of third diagonal portions.

In some examples, each third diagonal portion optionally extends between one of the outflow stitch points and one of the second intermediate stitch points.

In some examples, the plurality of inflow stitch points and the plurality of outflow stitch points optionally are circumferentially offset from each other.

In some examples, the plurality of first intermediate stitch points optionally are circumferentially aligned with the plurality of outflow stitch points.

In some examples, the plurality of second intermediate stitch points optionally are circumferentially aligned with the plurality of inflow stitch points.

In one of its basic methods, an embroidery patterning method for forming and assembling an outer skirt of a prosthetic valve comprises providing an automated sewing machine, which comprises a bearded needle and a looper. This basic method can preferably be provided with any one or more of the steps described elsewhere herein, in particular with those of the examples described hereafter. However, it should be understood that the basic method can preferably also be provided with any one or more of the steps shown in the figures and/or described in conjunction with the figures, either in addition to or alternatively to the steps of the examples described hereafter.

In some examples, the method comprises providing a multi-yarn thread, optionally extending through a draw hole of the looper.

In some examples, the method can comprise spreading a base layer in the automated sewing machine such that its inner surface is facing the looper and its outer surface is facing the bearded needle.

In some examples, the method comprises piercing the base layer by the bearded needle, optionally by lowering the needle from a top dead center towards the looper.

In some examples, the method comprises winding the multi-yarn thread around a shank of the bearded needle, optionally by continued lowering of the needle so as to insert a hooking portion of the needle inside a needle insertion hole of the looper, while rotating the looper in a first rotational direction.

In some examples, the method comprises hooking the multi-yarn thread by the hooking portion, optionally while elevating the bearded needle from the needle insertion hole.

In some examples, the method comprises forming a loop extending from a stitch point, optionally by further elevating the bearded needle and the multi-yarn thread hooked thereon through the base layer and to the dead center.

In some examples, the method can comprise stopping rotation movement of the looper in the first rotation direction.

In some examples, the method can comprise rotating the looper in a second rotational direction, optionally opposite to the first rotational direction.

In some examples, the method can comprise moving the base layer to position a subsequent stitch point between the bearded needle and the looper.

In some examples, the method can comprise repeating the steps of piercing the base layer, winding the multi-yarn thread, hooking the multi-yarn thread, forming a loop, stopping rotation movement of the looper, rotating the looper in a second rotational direction, and moving the base layer, one or more times, to form one or more additional loops at the subsequent one or more stitch points, thereby forming an outer portion of the multi-yarn thread optionally comprising a plurality of loops.

In some examples, the method can comprise attaching the base layer to a frame of a prosthetic valve, optionally such that the inner surface of the base layer is facing the frame.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

In the Figures:

FIG. 1A is perspective view of an exemplary mechanically expendable prosthetic valve.

FIG. 1B is a perspective view of the prosthetic valve of FIG. 1A.

FIG. 1C is a perspective view of the frame the prosthetic valve of FIGS. 1A-1B.

FIG. 2 shows an exemplary delivery assembly comprising a delivery apparatus carrying the prosthetic valve.

FIGS. 3A-3B show an outer skirt disposed over a frame, with an exemplary thread pattern disposed over the base layer of the skirt.

FIG. 4A shows another example of the outer skirt of FIGS. 3A-3B.

FIG. 4B shows an enlarged section of FIG. 4A.

FIGS. 5A-5B show an outer skirt disposed over a frame of, with another exemplary thread pattern disposed over the base layer of the skirt.

FIG. 6A shows another example of the outer skirt of FIGS. 5A-5B.

FIG. 6B shows an enlarged section of FIG. 6A.

FIG. 7A shows an exemplary outer skirt with a zig-zagging suture pattern disposed over the base layer.

FIG. 7B shows an exemplary outer skirt with a ring-like suture pattern extending around the base layer.

FIG. 7C shows an exemplary outer skirt with an X-shaped suture pattern disposed over the base layer.

FIG. 7D shows an exemplary outer skirt with the suture extending angularly around the circumference of the base layer.

FIG. 8A shows a lock-stitch formed at a stitch point in which a multi-yarn thread is laced with a backing thread.

FIG. 8B shows an enlarged section of FIG. 8A.

FIGS. 9A-9D illustrate some steps of a method for lock-stitch embroidering a multi-yarn thread to a base layer of a skirt.

FIG. 10 shows some components of an exemplary automated sewing machine.

FIGS. 11A-11F show exemplary multi-yarn thread patterns embroidered over base layers of outer skirts.

FIG. 12 shows a chain-stitch formed at a stitch point upon pulling of a multi-yarn thread through a base layer of a skirt.

FIGS. 13A-13D illustrate some steps of a method for chain-stitch embroidering a multi-yarn thread to a base layer of a skirt.

FIG. 14 shows a moss-stitch formed at a stitch point upon pulling of a multi-yarn thread through a base layer of a skirt.

FIGS. 15A-15D illustrate some steps of a method for moss-stitch embroidering a multi-yarn thread to a base layer of a skirt.

FIG. 16 shows a cross-sectional view of a portion of a skirt along which a series of moss-stitch loops are formed.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a,” “an,” and “the” include the plural forms unless the context clearly dictates otherwise. Additionally, the terms “have” or “includes” means “comprises”. Further, the terms “coupled”, “connected”, and “attached”, as used herein, are interchangeable and generally mean physically, mechanically, chemically, magnetically, and/or electrically coupled or linked and does not exclude the presence of intermediate elements between the coupled or associated items absent specific contrary language. As used herein, “and/or” means “and” or “or”, as well as “and” and “or”.

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the element. Directions and other relative references (e.g., inner and outer, upper and lower, above and below, left and right, and proximal and distal) may be used to facilitate discussion of the drawings and principles herein but are not intended to be limiting.

The terms “proximal” and “distal” are defined relative to the use position of a delivery apparatus. In general, the end of the delivery apparatus closest to the user of the apparatus is the proximal end, and the end of the delivery apparatus farthest from the user (e.g., the end that is inserted into a patient's body) is the distal end. The term “proximal” when used with two spatially separated positions or parts of an object can be understood to mean closer to or oriented towards the proximal end of the delivery apparatus. The term “distal” when used with two spatially separated positions or parts of an object can be understood to mean closer to or oriented towards the distal end of the delivery apparatus. The terms “longitudinal” and “axial” are interchangeable, and refer to an axis extending in the proximal and distal directions, unless otherwise expressly defined.

The terms “axial direction,” “radial direction,” and “circumferential direction” have been used herein to describe the arrangement and assembly of components relative to the geometry of the frame of the prosthetic valve, or the geometry of an inflatable balloon that can be used to expand a prosthetic valve. Such terms have been used for convenient description, but the disclosed examples are not strictly limited to the description. In particular, where a component or action is described relative to a particular direction, directions parallel to the specified direction as well as minor deviations therefrom are included. Thus, a description of a component extending along an axial direction of the frame does not require the component to be aligned with a center of the frame; rather, the component can extend substantially along a direction parallel to a central axis of the frame.

As used herein, the terms “integrally formed” and “unitary construction” refer to a construction that does not include any welds, fasteners, or other means for securing separately formed pieces of material to each other.

As used herein, operations that occur “simultaneously” or “concurrently” occur generally at the same time as one another, although delays in the occurrence of operation relative to the other due to, for example, spacing between components, are expressly within the scope of the above terms, absent specific contrary language.

As used herein, terms such as “first,” “second,” and the like are intended to serve as respective labels of distinct components, steps, etc. and are not intended to connote or imply a specific sequence or priority. For example, unless otherwise stated, a step of performing a second action and/or of forming a second component may be performed prior to a step of performing a first action and/or of forming a first component.

As used herein, the term “substantially” means the listed value and/or property and any value and/or property that is at least 75% of the listed value and/or property. Equivalently, the term “substantially” means the listed value and/or property and any value and/or property that differs from the listed value and/or property by at most 25%. For example, “at least substantially parallel” refers to directions that are fully parallel, and to directions that diverge by up to 22.5 degrees.

In the present disclosure, a reference numeral that includes an alphabetic label (for example, “a,” “b,” “c,” etc.) is to be understood as labeling a particular example of the structure or component corresponding to the reference numeral. Accordingly, it is to be understood that components sharing like names and/or like reference numerals (for example, with different alphabetic labels or without alphabetic labels) may share any properties and/or characteristics as disclosed herein even when certain such components are not specifically described and/or addressed herein.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

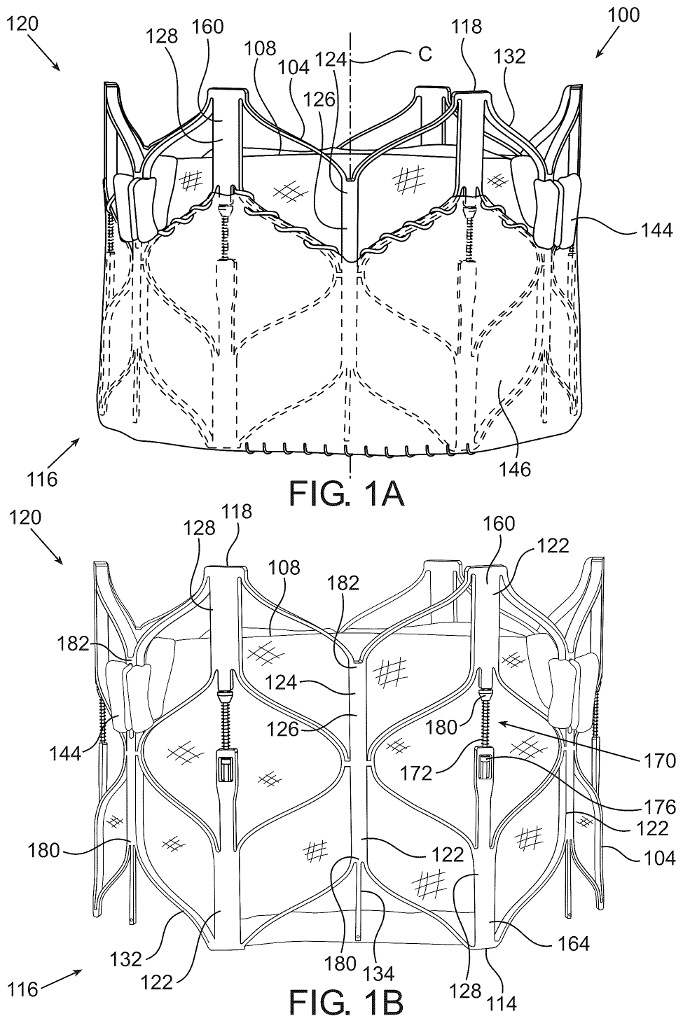

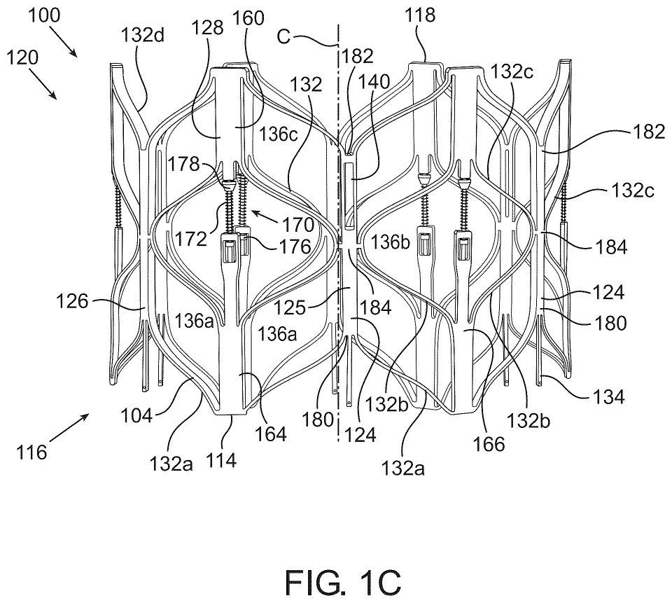

FIGS. 1A-1C illustrate a prosthetic valve 100, according to one example. The prosthetic valve 100 can be configured to replace a native heart valve (e.g., aortic, mitral, pulmonary, and/or tricuspid valves). The prosthetic valve 100 is illustrated as a mechanically expandable prosthetic valve that can be radially compressed for delivery to an implantation location within a patient's body and then radially expanded to a working diameter at the implantation location. The prosthetic valve 100 can include a frame 104 having an annular shape. The prosthetic valve 100 can optionally further include a valvular structure 108 supported within and coupled to the frame 104. FIG. 1A shows a perspective view of a prosthetic valve 100 including a skirt 150 and a valvular structure 108 thereof. FIG. 1B shows the prosthetic valve 100 of FIG. 1A with the skirt 150 removed from view. FIG. 1C shows the frame 104 of the prosthetic valve 100 of FIGS. 1A-1B without any soft components, such as a skirt or a valvular structure.

In the example, the valvular structure 108 includes one or more leaflets 112 made of flexible material and configured to open and close to regulate blood flow. In one example, the valvular structure 108 can have three leaflets 112, which can optionally be arranged to collapse in a tricuspid arrangement. The leaflets 112 can optionally be made in whole or in part from pericardial tissue (e.g., bovine pericardial tissue), biocompatible synthetic materials, or various other suitable natural or synthetic materials.

As illustrated, the frame 104 has an inflow end 116, an outflow end 120, and a central longitudinal axis C extending in a direction from the inflow end 116 to the outflow end 120. The frame 104 can optionally include a plurality of vertical posts 122 aligned with the central longitudinal axis C. The vertical posts 122 can optionally include support posts 124 and actuation posts 128 spaced along a circumference of the frame 104. In one example, the support posts 124 and actuation posts 128 can optionally be arranged in an alternating manner along the circumference of the frame 104. The frame 104 can optionally further include a plurality of angled struts 132 extending circumferentially between adjacent support posts 124 and actuation posts 128 and interconnecting the support posts 124 and actuation posts 128. The angled struts 132, support posts 124, and actuation posts 128, define cells 136 of the frame 104. As illustrated, the angled struts 132 can have a curved shape.

One or more commissure windows 140 can optionally be formed in one or more of the support posts 124. Commissures 144 can optionally be formed at the commissure windows 140 to couple the leaflets 112 to the frame 104. Support posts 124 can optionally include commissure support posts 125, which are support posts 124 that include commissure windows 140, and non-commissural support posts 126, which are support posts 124 devoid of commissure windows. The commissure support posts 125 and non-commissural support posts 126 can optionally be arranged in an alternating manner along the circumference of the frame 104. In the illustrated example, frame 104 includes a total of six support posts 124, three of which are commissure support posts 125 and three of which are non-commissural support posts 126.

One or more of the support posts 124 can optionally further include cantilevered struts 134 extending from the corresponding post inflow ends 180. In some examples, the cantilevered struts 134 can optionally extend such that distal ends of the cantilevered struts 134 align with or substantially align with the inflow end 116 of the frame 104.

The prosthetic valve 100 can optionally further include one or more skirts or sealing members. For example, the prosthetic valve 100 can optionally include an inner skirt (not shown), mounted on the radially inner surface of the frame 104. The inner skirt can function as a sealing member to prevent or decrease perivalvular leakage, to anchor the leaflets 112 to the frame 104, and/or to protect the leaflets 112 against damage caused by contact with the frame 104 during crimping and during working cycles of the prosthetic valve 100. The prosthetic valve 100 can optionally further include an outer skirt 150 mounted on the outer surface of the frame 104. The outer skirt 150 can function as a sealing member for the prosthetic valve 100 by sealing against the tissue of the native valve annulus and helping to reduce paravalvular leakage (PVL) past the prosthetic valve 100. The inner and outer skirts can be formed from any of various suitable biocompatible materials, including any of various synthetic materials, including fabrics (e.g., polyethylene terephthalate fabric) or natural tissue (e.g., pericardial tissue). Further details regarding the use of skirts or sealing members in prosthetic valves can be found, for example, in U.S. Patent Application No. 62/854,702 and PCT Patent Application No. US2020/024559, each of which is incorporated by reference herein.

In some cases, as shown in the example illustrated in FIG. 1B, inflow edge portions of the leaflets 112 can optionally be attached to the cantilevered struts 134 and/or to selected struts 132 of the frame 104. Alternatively or additionally, cantilevered struts 134 can prevent or mitigate portions of an outer skirt 150 from extending radially inwardly and thereby prevent or mitigate any obstruction of flow through the inflow end 116 of the frame 104 caused by the outer skirt 150. The cantilevered struts 134 can further serve as supports to which portions of the inner and/or outer skirts can be coupled, as shown in FIG. 1A. For example, sutures used to connect the inner and/or outer skirts can be wrapped around the cantilevered struts 134 and/or can extend through apertures formed at end portions of the cantilevered struts 134.

In some implementations, leaflets 112 can be sutured directly to angled struts 132 of the frame 104. In other implementation, inflow edge portions of the leaflets can optionally be sutured to an inner skirt generally along the scallop line. Optionally, the inner skirt can in turn be sutured, via one or more sutures, for example, to adjacent angled struts 132 of the frame 104.

Each support post 124, including any commissure support posts 125 or non-commissural support post 126, extends between a post inflow end 180 which is closer to the inflow end 116 of the valve 100, and a post outflow end 182 which is closer to the outflow end 120 of the valve 100. Two angled struts 132 intersect with each support post 124 at a post inflow end 180, which is circumferentially disposed between two adjacent inflow apices 114, such that a corresponding cantilevered strut 134 can optionally extend distally from the post inflow end 180. Similarly, two angled struts 132 intersect with each support post 124 at a post outflow end 182, which is circumferentially disposed between two adjacent outflow apices 118.

As further shown, each commissure support post 125 and each non-commissural support post 126 also intersects, at a middle portion thereof, with four additional angled struts 132 extending from adjacent upper post members 160 and lower post members 164 on both sides, resulting in each support posts 124, and specifically, each commissure support post 125 and each non-commissural support post 126, intersecting with a total of at least eight curved struts extending from adjacent actuation posts 128.

In one example, the frame 104 can be adjusted between a radially expanded configuration and a radially compressed configuration by deflecting the angled struts 132. In one example, the frame 104 (e.g., the posts and struts) can optionally be made of biocompatible plastically-expandable materials that will allow the frame 104 to be adjusted between the radially expanded configuration and radially compressed configuration. Suitable examples of plastically-expandable materials that can be used in forming the frame 104 include, but are not limited to, stainless steel, cobalt-chromium alloy, and/or nickel-titanium alloy (which can also be referred to as “NiTi” or “nitinol”).

In some examples, one or more actuators 170 can optionally be coupled to the actuation posts 128, and used to adjust the frame 104 between the radially expanded configuration and the radially compressed configuration. In one example, each actuation post 128 can optionally include an upper post member 160 and a lower post member 164 (the terms “upper” and “lower” are relative to the orientation of the prosthetic valve 100 in FIGS. 1A-1C) aligned with the longitudinal axis C and having opposing ends separated by a gap. The respective actuator 170 can optionally be coupled to the post members 160, 164 and operable to increase or decrease the gap therebetween in order to radially compress or expand the frame 104. Angled struts 132 can converge with upper post members 160 to define outflow apices 118 at the outflow end 120. Angled struts 132 can optionally similarly converge with lower post members 164 to define inflow apices 114 at the inflow end 116.

In one example, the actuator 170 can optionally include an actuator rod 172 with an attached actuator head. In the example illustrated in FIGS. 1A-1C, the actuator rod 172 extends through or into the post members 160, 164 and across the gap therebetween. In the example illustrated in FIGS. 1A-1C, the actuator rod 172 is inserted into the upper post member 160 from the outflow end 120, and the actuator head (hidden from view in FIGS. 1A-1C) can optionally be disposed or retained at the outflow apex 118 of the upper post member 160.

In some examples, the actuator rod 172 is externally threaded. As illustrated in FIGS. 1A-1C, the lower post member 164 can optionally include a nut 176 with an internal thread to threadedly engage the actuator rod 172. In this case, the actuator rod 172 can optionally be axially translated by rotating the actuator rod 172 relative to the nut 176. In some examples, the actuator rod 172 can optionally be freely slidable relative to the upper post member 160. In other examples, the actuator rod 172 can optionally threadedly engage the upper post member 160. The term “axially translated”, as used herein, refers to translation along an axis coinciding with or parallel to the central longitudinal axis C.

In one scenario, the actuator rod 172 can optionally be rotated in a first direction to move the upper post member 160 towards the lower post member 164 and thereby decrease the size of the gap therebetween, which can have the effect of radially expanding the frame 104. In another scenario, the lower post member 164 may be held steady while the actuator rod 172 is rotated in a second direction to move the upper post member 160 away from the lower post member 164 and thereby increase the size of the gap therebetween, which can have the effect of radially compressing the frame 104.

The actuator rod 172 also can optionally include a stopper 178 (e.g., in the form of a nut, washer or flange) disposed thereon. The stopper 178 can optionally be disposed on actuator rod 172 such that it sits within the gap therebetween. Further, the stopper 178 can optionally be integrally formed on or fixedly coupled to the actuator rod 172 such that it does not move relative to the actuator rod 172. Thus, the stopper 178 can remain in a fixed axial position on the actuator rod 172 such that it moves in lockstep with the actuator rod 172.

When the actuator rod 172 is rotated in a direction configured to collapse the prosthetic valve, the stopper 178 moves toward the outflow end 120 of the frame until the stopper 178 abuts the inflow end of the upper post member 160. Upon further rotation of the actuator rod 172, the stopper 178 can apply a proximally directed force to the upper post member 160 to radially compress the frame 104. Specifically, during crimping/radial compression of the prosthetic valve 100, the actuator rod 172 can optionally be rotated in a direction that causes the stopper 178 to push against (i.e., provide a proximally directed force to) the inflow end of the upper post member 160, thereby causing the upper post member 160 to move away from the lower post member 164, and thereby axially elongating and radially compressing the prosthetic valve 100.

In an alternative implementation, some of the actuator rods 172 can optionally be rotated in one direction while the other actuator rods 172 are rotated in an opposite direction simultaneously to either radially expand the frame or radially compress the frame. This counter-rotation of the actuator rods can be used to help reduce the likelihood of the entire frame 104 rotating about the central longitudinal axis C during rotation of the actuator rods 172 about their respective axes (e.g., when radially expanding the frame 104).

Additional examples of mechanically expandable valves can be found in International Application No. PCT/US2021/052745 and U.S. Provisional Applications Nos. 63/209,904 and 63/282,463, which are incorporated by reference herein.

As shown, the actuation posts 128 are arranged in pairs, each pair including an upper post member 160 and a lower post member 164 which can optionally be axially aligned with each other, and each pair of actuation posts 128 can optionally be connected, such as via angled struts 132, to a commissure support post 125 on one side thereof, and to a non-commissural support post 126 on the other side.

As further shown in FIG. 1C, the plurality of angled struts can optionally include four rungs of struts. A rung of first angled struts 132a is arranged at the inflow end 116 of the frame, wherein the first angled struts 132a extend proximally from the inflow apices. A rung of fourth angled struts 132d is arranged at the outflow end 120 of the frame, wherein the fourth angled struts 132d extend distally from the outflow apices 118. A rung of second angled struts 132b is arranged proximal to the rung of first angled struts 132a, wherein the second angled struts 132b are parallel to the corresponding first angled struts 132a. A rung of third angled struts 132c is arranged distal to the rung of first angled struts 132d, wherein the third angled struts 132c are parallel to the corresponding fourth angled struts 132c. The plurality of cells 136 can similarly define inflow cells 136a extending between the first 132a and second 132b angled struts, outflow cells 136c extending between the third 132c and fourth 132d angled struts, and intermediate cells 136b defined between the second 132b and third 132c angled struts.

Each lower post member 164 defines a lower post junction 166 at the intersection between the lower post member 164 and the second angled struts 132b. Each support post 126 defines a post intermediate junction 184, at the intersection of the support post 126 with the second angled struts 132b and third angled struts 132c.

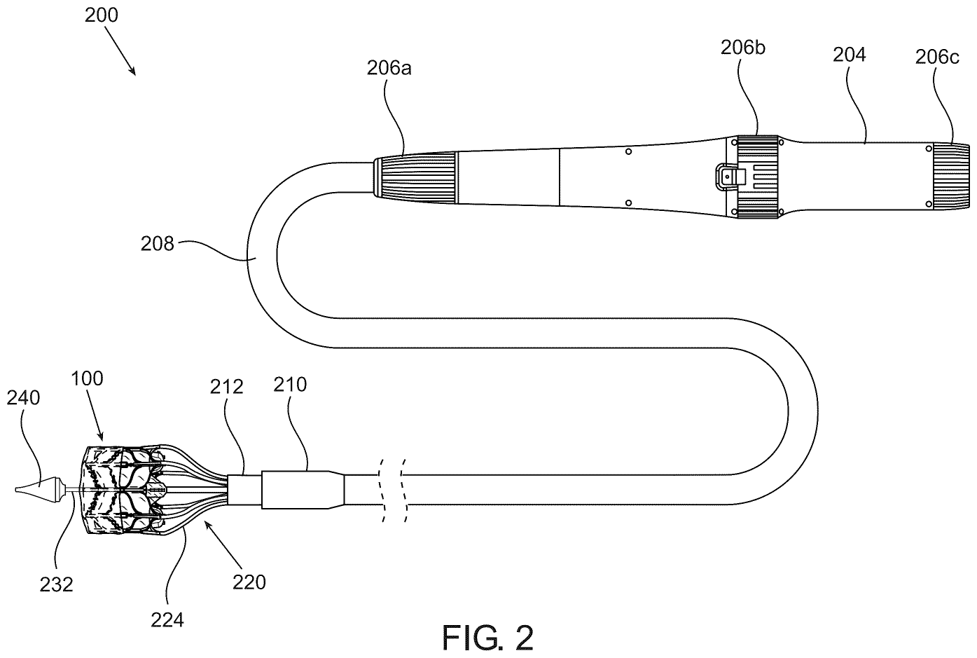

FIG. 2 illustrates a delivery apparatus 200, according to one configuration, adapted to deliver a mechanically expandable prosthetic valve 100 described herein. The prosthetic valve 100 can optionally be releasably coupled to the delivery apparatus 200. It should be understood that the delivery apparatus 200 can optionally be used to implant prosthetic devices other than prosthetic valves, such as stents or grafts.

The delivery apparatus 200 in the illustrated example generally includes a handle 204, an outer elongated shaft 208 extending distally from the handle 204 and at least one actuator assembly 220 extending distally through the outer shaft 208. The delivery apparatus 200 can optionally also include an elongated nosecone shaft 232 extending distally from the handle 204 through the outer shaft 208. A nosecone 240 can optionally be connected to the distal end of the nosecone shaft 232. The at least one actuator assembly 220 can optionally be configured to radially expand and/or radially collapse the prosthetic valve 100 when actuated.

As illustrated, one actuator assembly 220 can optionally be provided for each actuator 170 on the prosthetic valve 100. For example, six actuator assemblies 220 can optionally be provided for a prosthetic valve 100 having six actuators. In other configurations, however, any greater or fewer number of actuator assemblies can be present.

The distal end portion of the shaft 208 can optionally be sized and shaped to house the prosthetic valve 100 in a radially compressed, delivery state during delivery of the prosthetic valve through, for example, the vasculature of a patient. In this way, the distal end portion of shaft 208 functions as a delivery sheath or capsule for the prosthetic valve during delivery.

The actuator assemblies 220 can optionally be releasably coupled to the prosthetic valve 100. For example, in the illustrated configuration, each actuator assembly 220 can optionally be coupled to a respective actuator of the prosthetic valve 100. Each actuator assembly 220 can optionally comprise a support tube 224, an actuator member 226 (concealed from view within support tube 224 in FIG. 2), and optionally a locking tool. When actuated, the actuator assembly can transmit pushing and/or pulling forces to portions of the prosthetic valve to radially expand and collapse the prosthetic valve as previously described. The actuator assemblies 220 can optionally be at least partially disposed radially within, and extend axially through, one or more lumens of the outer shaft 208. For instance, the actuator assemblies 220 can optionally extend through a central lumen of the shaft 208 or through separate respective lumens formed in the shaft 208.

The terms “releasably coupled” or “releasably attached”, as used herein, are interchangeable, and refer to two components coupled in such a way that they are coupled together and can be separated without plastically deforming either of the components.

Although not illustrated, the delivery apparatus 200 can optionally include, in some implementations, a multi-lumen delivery shaft 212 extending through the lumen of the outer shaft and having a plurality of lumens therein. Any of the nosecone shaft 232 and/or actuation assemblies 220 can optionally extend through lumens of the multi-lumen delivery shaft 212.

The actuator member 226 of each actuator assembly 220 can optionally be releasably coupled to a respective actuator 170 of the prosthetic valve. The support tube 224 of each actuator assembly 220 can optionally abut an adjacent portion of the frame of the prosthetic valve, such as an outflow apex 118. In this manner, during valve expansion, the support tubes 224 can prevent movement of the outflow end of the prosthetic valve relative to the delivery apparatus while the actuator members of the actuator assemblies 220 can actuate the actuators of the prosthetic valve and cause the inflow end of the prosthetic valve to move toward the outflow end of the prosthetic valve.

The handle 204 of the delivery apparatus 200 can optionally include one or more control mechanisms (e.g., knobs 206 or other actuating mechanisms) for controlling different components of the delivery apparatus 200 in order to expand and/or deploy the prosthetic valve 100. For instance, in the illustrated example, the handle 204 comprises first, second, and third knobs 206a, 206b, and 206c.

The first knob 206a can optionally be a rotatable knob configured to produce axial movement of the outer shaft 208 relative to the prosthetic valve 100 in the distal and/or proximal directions in order deploy the prosthetic valve from the delivery sheath once the prosthetic valve has been advanced to a location at or adjacent the desired site of implantation within a patient. For instance, rotation of the first knob 206a in a first direction (e.g., clockwise) can retract the outer shaft 208 proximally relative to the prosthetic valve 100 and rotation of the first knob 206a in a second direction (e.g., counterclockwise) can advance the outer shaft 208 distally. In other configurations, the first knob 206a can optionally be actuated by sliding or moving the knob 206a axially, such as puling and/or pushing the knob. In still further configurations, actuations of the first knob 206a, such as by rotation or sliding the first knob 206a, can produce axial movement of the actuator assemblies 220 and thereby the prosthetic valve 100 relative to the delivery sheath to advance the prosthetic valve distally from the sheath.

In one example, a capsule 210 can optionally be attached to a distal end of the outer shaft 208. Axial movement of the outer shaft 208 in a distal direction relative to the other shafts and prosthetic valve can move the capsule 210 over the distal end portions of the actuation assemblies 220 and the prosthetic valve 100 such that the prosthetic valve 100 is enclosed within the capsule 210. Axial movement of the outer shaft 208 in a proximal direction relative to the other shafts and the prosthetic valve can retract the capsule 210 from the prosthetic valve 100, exposing the prosthetic valve, for example, for deployment at an implantation location.

The second knob 206b can optionally be a rotatable knob configured to produce radial expansion and/or contraction of the prosthetic valve 160. For instance, rotation of the second knob 306b can optionally move the actuator members and the support tubes 224 of actuator assemblies 220 axially relative to one another. The actuator members or drivers of actuator assemblies 220 in turn cause corresponding movement of the actuators 170 of the prosthetic valve. Rotation of the second knob 206b in a first direction (e.g., clockwise) can radially expand the prosthetic valve 100 and rotation of the second knob 206b in a second direction (e.g., counterclockwise) can radially collapse the prosthetic valve 100. In other configurations, the second knob 206b can be actuated by sliding or moving the knob 206b axially, such as pulling and/or pushing the knob.

The third knob 206c can optionally be a rotatable knob configured to retain the prosthetic valve 100 in an expanded state. For instance, the third knob 206c can optionally be operatively connected to a proximal end portion of the locking tool of each actuator assembly 220. Rotation of the third knob 206c in a first direction (e.g., clockwise) can rotate each locking tool to advance the locking nuts to their distal positions to resist radial compression of the frame of the prosthetic valve. Rotation of the knob 206c in the opposite direction (e.g., counterclockwise) can rotate each locking tool in the opposite direction to decouple each locking tool from the prosthetic valve 100. In other configurations, the third knob 206c can optionally be actuated by sliding or moving the third knob 206c axially, such as pulling and/or pushing the knob. In some examples, the prosthetic valve can optionally be self-locking, in which case a locking tool is not required. For example, the frame of the prosthetic valve can optionally include locking features that automatically engage the actuator members of the prosthetic valve to resist radial compression of the prosthetic valve after it is expanded, such as disclosed in U.S. Application Nos. 63/085,947, 63/138,599, and 63/179,766.

Although not shown, the handle 204 can optionally include a fourth rotatable knob operative connected to a proximal end portion of each actuator member. The fourth knob can optionally be configured to rotate each actuator member, upon rotation of the knob, to unscrew each actuator member 226 from the proximal portion of a respective actuator. As described above, once the locking tools and the actuator members are uncoupled from the prosthetic valve 100, they can optionally be removed from the patient.

In some examples, the delivery apparatus 200 with the prosthetic valve 100 assembled thereon, can optionally be packaged in a sterile package that can be supplied to end users for storage and eventual use. In some examples, the leaflets of the prosthetic valve (typically made from bovine pericardium tissue or other natural or synthetic tissues) are treated during the manufacturing process so that they are completely or substantially dehydrated and can be stored in a partially or fully crimped state without a hydrating fluid. In this manner, the package containing the prosthetic valve 100 and the delivery apparatus 200, respectively, can be free of any liquid. Methods for treating tissue leaflets for dry storage are disclosed in U.S. Pat. Nos. 8,007,992 and 8,357,387, both of which documents are incorporated herein by reference.

In some cases, the external peripheral surface of an implanted prosthetic valve can be in discontinuous engagement with the inner surface of the surrounding anatomical wall (e.g., annular wall), which may result in a lack of appropriate sealing therebetween. Gaps in the form of voids or channels can be formed around the periphery of the prosthetic valve due to the fact that the inner surface of the surrounding arterial or annular wall may have an irregular surface shape, while the outer surface of the prosthetic heart valve is typically circular, and therefore may cause paravalvular leakage (PVL) around the valve.

Paravalvular leakage (PVL) is a complication that is related to the implantation of prosthetic heart valves. It may occur when blood flows through a channel or gap located between the structure of an implanted prosthetic heart valve in an expanded state and the site of implantation (e.g., the cardiac or arterial tissue surrounding it), due to a lack of appropriate sealing therebetween. PVL has been previously shown to greatly affect the clinical outcome of transcatheter aortic valve implantation procedures, and the severity of PVL has been correlated with patient mortality.

In order to address this issue, adaptive seal components can be provided around the external peripheral surface of the prosthetic heart valve, in order to provide improved sealing thereto, as previously disclosed, for example, in U.S. Pat. No. 10,722,316, which is incorporated herein by reference. Typically, these seal components (also known as external skirts, or PVL skirts) can be configured to improve PVL sealing around the implanted prosthetic heart valves. In addition, several PVL skirts were designed to promote tissue ingrowth (for example, utilizing textured yarns over the external surface of the skirt).

Sealing members which comprise a first tear resistant layer and a second cushioning layer attached thereto and extending radially outward therefrom have been previously disclosed, for example in US Pub. No. 2019/0374337 which is incorporated herein by reference. US Pub. No. 2019/0374337 discloses a second layer comprising pile strands or pile yarns woven or knitted into loops attached to the first layer. Such strands or yarns may be spaced from each other, in a manner that can encourage tissue ingrowth. Thus, for applications in which tissue ingrowth is to be avoided, it may be preferable to form a second layer from a continuous material that is devoid of strands and yarns that can be interspaced from each other.

An important design parameter of a transcatheter prosthetic heart valve is the diameter of the compressed or crimped profile. The diameter of the crimped profile is important because it directly influences the physician's ability to advance the transcatheter prosthetic heart valve through the femoral artery or vein. More particularly, a smaller profile allows for treatment of a wider population of patients, with enhanced safety. However, having the entire outer surface of an outer skirt extending radially outwards, can increase the overall diameter of the crimped profile. International Patent Application Pub. No. WO 2021/202450 discloses fuzzy or puffy sutures that can be sutured at selected patterns through a base layer of the outer skirt. However, manually passing expandable sutures through the fabric of the outer skirt is a tedious and time consuming procedure.

The current specification provides several fabrication procedures and sealing members resulting from such procedures that may address such challenges.

In some examples, there is provided a skirt that includes a base layer disposed around the frame of a prosthetic valve, and at least one multi-yarn thread extending in a pattern over the outer surface of the base layer. A thread termed to extend in a pattern over the base layer refers to the thread extending along a path that can include one or more linear or curved portions, without covering the entire outer surface of the base layer.

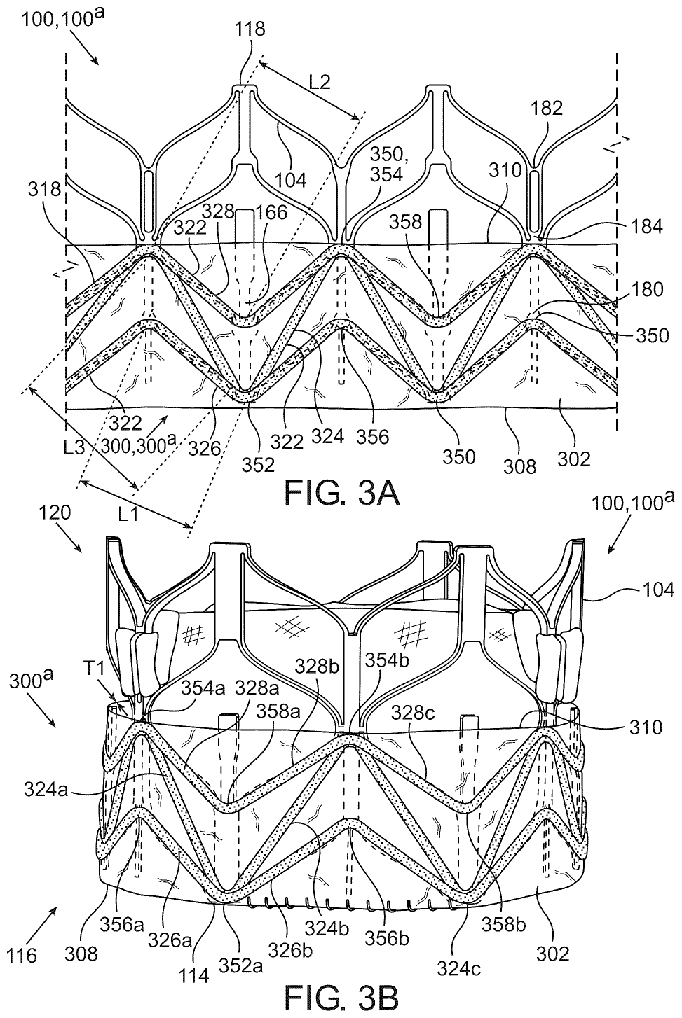

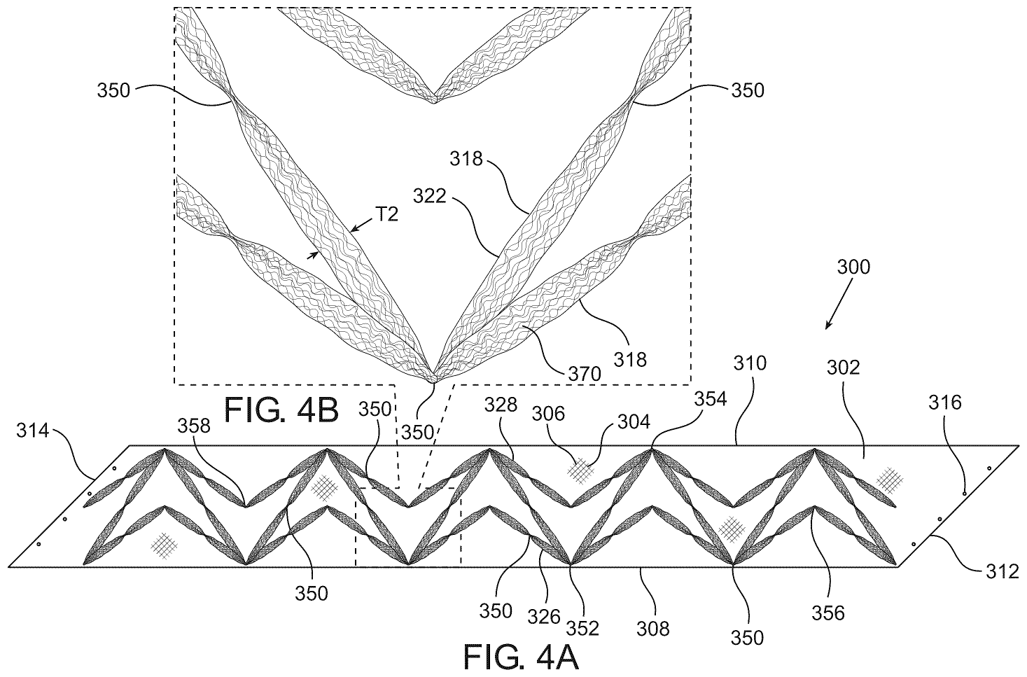

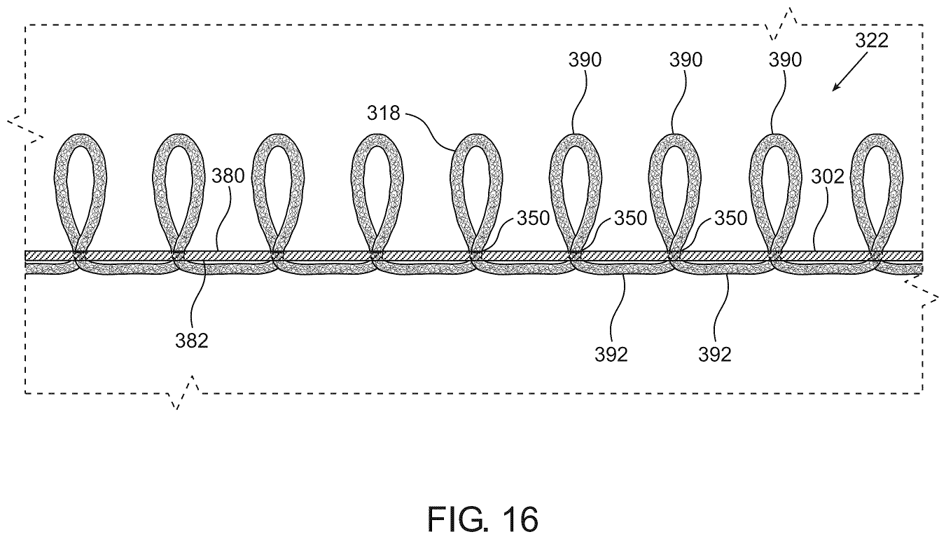

FIGS. 3A-4 show an outer skirt 300 comprising a multi-yarn thread 318 extending over the outer surface 380 of a base layer 302 thereof. Base layer 302 defines an inner surface 382 oriented radially inward (see, e.g., FIG. 16), facing the frame 104 and the longitudinal axis C, and an opposite outer surface 380 oriented radially outward, facing away from the frame 104 and the longitudinal axis C, when the outer skirt 300 is disposed around, and coupled to, the frame 104. Thus, any exemplary outer skirt 300 can optionally be coupled to the frame 140 in the same manner described above with respect to outer skirt 150. Any reference to a multi-yarn thread 318 extending over a base layer 302 throughout the current disclosure, may refer to a single multi-yarn thread 318 that can be stitched to the base layer 302 at a plurality of stitch points 350, or a plurality of multi-yarn threads 318 stitched to the base layer 350 at stitch points 350, forming together the desired pattern over the base layer 302.

FIG. 3A shows an exemplary outer skirt 300 disposed over a frame 104 of valve 100 in a flattened configuration, and FIG. 3B shows a view in perspective of the valve 100 with outer skirt 300 of FIG. 3A. FIG. 4A shows an exemplary skirt 300 in a flattened configuration, with a zoomed-in view on a portion of the skirt 302 comprising portions of breaded threads 318 disposed over the outer surface 380 of the base layer 302. FIG. 4B shows an enlarged section of FIG. 4A in the vicinity of an inflow stitch point 352.

As shown in FIGS. 4A-4B, the base layer 302 can optionally extend from an inflow edge portion 308 to an outflow edge portion 310, wherein the inflow edge portion 308 of the base layer 302 can be closer to the inflow end 116, while the outflow edge portion 310 of base layer 302 is disposed between the inflow end 116 and outflow end 120, when arranged around the frame 104 (skirt coupled to frame shown for example in FIG. 3B). In the flattened unassembled view of the skirt 300 in FIGS. 4A-4B, the base layer further comprises a first side edge portion 312, and an opposite second side edge portion 314, each of which extends between the inflow 308 and outflow 312 edge portions of the base layer 302.

In some examples, the first and second side edge portions 312, 314 can optionally be non-perpendicular to the inflow edge portion 308. For example, the first and second edge portions 312, 314 can optionally extend at angles of about 45 degrees (or in a range of 40 to 50 degrees) relative to the inflow edge portion 308. Therefore, an overall general shape of the outer skirt 300 can optionally be that of a rhomboid or parallelogram.

Optionally, the first and second side edge portions 312, 314 can each comprise a plurality of apertures 316 extending therethrough. Thus, when the outer skirt 300 is converted into its annular configuration (e.g., when mounted around the frame 104 a prosthetic valve 100, as shown in FIG. 3B for example), the first and second side edge portions 312, 314 can optionally overlap one another with their respective apertures 316 overlapping as well. A suture (not explicitly shown) can then be used to form a plurality of stitches in and in-and-out pattern through the overlapping apertures 316, thereby securing the first and second edge portions 312, 314 together and forming the annular configuration of the outer skirt 300.

While both the inflow edge portion 308 and outflow edge portion 310 are shown to be parallel to each other, and relatively straight in the circumferential direction, it is to be understood that any of these edges can optionally be non-linear. For instance, in some examples (not shown) an outflow edge portion of the base layer can optionally be formed to include a plurality of projections that can define an undulating shape, which can, in some cases, better track the contour of the struts of some types of frames the skirt 300 can be attached to. The term “circumferential direction”, as used herein, refers to a direction following the circumference or perimeter of a valve. A reference to a component of a skirt 300 extending in a circumferential direction, refers to such a direction when the skirt 300 is disposed around the frame of the valve.

When the outer skirt 300 is arranged around an outer surface of the frame of the prosthetic device (e.g., as shown in FIG. 3B) sutures or other couplers can optionally be used to secure any of the inflow edge portion 308 and/or outflow edge portion 310 to the struts of the frame, and/or to other components of the prosthetic valve, such as an inner skirt that can optionally be disposed around an inner surface of the frame, and/or leaflets of the prosthetic valve.

In some examples, the base layer 302 can optionally be a single layer of fabric. In some examples, the base layer 302 can optionally be a woven layer comprising a plan weave pattern having two sets if interwoven yarns, configured as warp fibers 304 and weft fibers 306. The warp fibers 304 can optionally extend parallel to the first and second side edge portions 312, 314, and the weft fibers 306 can optionally extend in a direction perpendicular to the warp fibers 304. In some examples, the density of the warp fibers 304 can be from about 10 fibers per inch to about 200 fibers per inch, about 50 fibers per inch to about 200 fibers per inch, or about 100 fibers per inch to about 200 fibers per inch. While warp 304 and weft 306 fibers are illustrated in FIG. 4A, it is to be understood that this is shown by way of illustration and no limitation, and that in some examples, the base layer 302 is not necessarily a woven layer.

As mentioned above, the outer skirt 300 comprises one or more multi-yarn threads 318 attached to and disposed over the base layer 302, such that one or more outer portions 322 of the multi-yarn thread(s) 318 extend over, and radially outward to, the base layer 302. An outer portion 322 is defined as any continuous portion of the braded thread 320 extending between two stitch points 350. A stitch point 350 is defined as a point or a region at which the multi-yarn thread is stitched or otherwise attached to the base layer 302. An outer portion 322 extending between two stitch points 350 can optionally include one or more additional stitch points 350 through which it is attached to the base layer 350, such that an outer portion 322 can be further divided into two or more shorter outer portions 350, which can optionally be continuous with each other.

Various exemplary implementations for outer skirt 300 and prosthetic valves 100 comprising such skirts attached to their frames, can be referred to, throughout the specification, with superscripts, for ease of explanation of features that refer to such exemplary implementations. It is to be understood, however, that any reference to structural or functional features of any device or component, without a superscript, refers to these features being commonly shared by all specific exemplary implementations that can be also indicated by superscripts. In contrast, features emphasized with respect to an exemplary implementation of any device or component, including outer skirt 300, referred to with a superscript, may be optionally shared by some but not necessarily all other exemplary implementations.

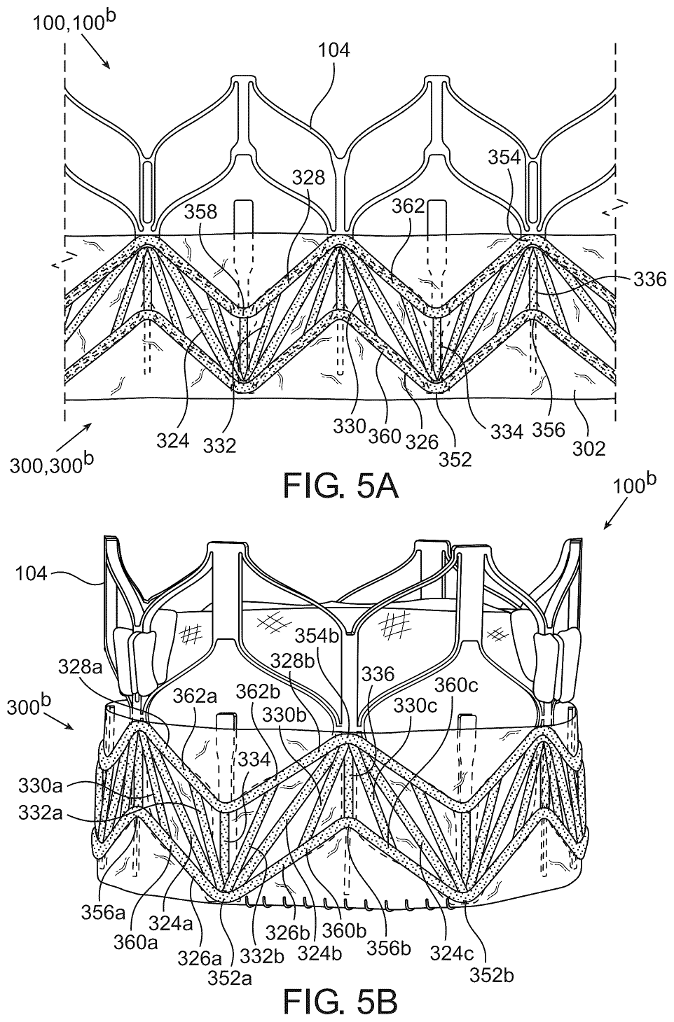

FIGS. 3A-3B show a prosthetic valve 100^{a} comprising an exemplary outer skirt 300^{a}. The prosthetic valve 100^{a} can include a frame 104 and valvular structure 108 as previously described above with respect to FIGS. 1A-1C, and in the interest of brevity will not be described further. While a mechanically-expandable frame is illustrated for exemplary valve 100^{a} in FIGS. 3A-3B, as well as for other exemplary valves described below (such as prosthetic valve 100^{c} shown in FIGS. 5A-5B), it is to be understood that this is not meant to be limiting, and that any of the exemplary outer skirts 300 described throughout the specification can optionally be used in combination with the frame of any other type of prosthetic valve, such as balloon-expandable valves, self-expandable valves, mechanically-expandable valves operable by actuation mechanisms that can be different than those described above with respect to FIGS. 1A-2, or a hybrid mechanically-expandable and self-expandable valve.

Outer skirt 300^{a} is an exemplary implementation of outer skirt 300, and thus includes all of the features described for outer skirt 300 throughout the current disclosure, except for the pattern of the braded thread(s) 318 disposed over the base layer 302 as elaborated in further detail below. Multi-yarn thread 318 of outer skirt 300^{a} defines a plurality of outer patterns 322 disposed between stitch points 350 that include: inflow stitch points 352, outflow stitch points 354, first intermediate stitch points 356, and second intermediate stitch points 358. Each group of stitch points 350 relates to a plurality of stitch points 350 which are disposed at the same level between the inflow and outflow edge portion 308, 310 of the base layer 302, and are circumferentially spaced from each other.

Inflow stitch points 352 are closer to the inflow edge portion 308 relative to other stitch points 350, and can optionally be at or proximate to the inflow edge portion 308 of base layer 302. Outflow stitch points 354 are closer to the outflow edge portion 310 relative to other stitch points 350, and can optionally be at or proximate to the outflow edge portion 310 of base layer 302. The outflow 354 and inflow 352 stitch points are both axially and circumferentially offset from each other, such that each outflow stitch point 354 is circumferentially positioned between the circumferential positions of adjacent inflow stitch points 352, and each inflow stitch point 352 is similarly circumferentially positioned between the positions of adjacent outflow stitch points 354. In some examples, the inflow stitch points 352 are equally spaced from each other in the circumferential direction. In some examples, the outflow stitch points 354 are equally spaced from each other in the circumferential direction. In some examples, each outflow stitch point 354 is equally spaced from adjacent inflow stitch points 352 on both sides. In some examples, each inflow stitch point 352 is equally spaced from adjacent outflow stitch points 354 on both sides.

First intermediate stitch points 356 are proximal to the inflow stitch points 352, and are circumferentially aligned with the outflow stitch points 354. Second intermediate stitch points 358 are distal to the outflow stitch points 354, and are circumferentially aligned with the inflow stitch points 352. In some examples, the first intermediate stitch points 356 are equally spaced from each other in the circumferential direction. In some examples, the second intermediate stitch points 358 are equally spaced from each other in the circumferential direction.

The outer portions 322 of multi-yarn thread(s) 318 of outer skirt 300^{a} include first diagonal portions 324, second diagonal portions 326, and third diagonal portions 328. Each first diagonal portion 324 extends between an inflow stitch point 352 and an outflow stitch point 354. Since the inflow 352 and outflow 354 stitch points are circumferentially offset from each other, the first diagonal portions 324 are angled with respect to the longitudinal axis C.

Each second diagonal portion 326 extends between an inflow stitch point 352 and a first intermediate stitch point 356 along a length L1, as indicated in FIG. 3A. Since the inflow 352 and first intermediate 356 stitch points are circumferentially offset from each other, the second diagonal portions 326 are angled with respect to the longitudinal axis C. Each third diagonal portion 328 extends between an outflow stitch point 354 and a second intermediate stitch point 358 along a length L2. Since the outflow 354 and second intermediate 358 stitch points are circumferentially offset from each other, the third diagonal portions 328 are angled with respect to the longitudinal axis C.

In some examples, an individual second diagonal portion 326 can optionally be parallel to a corresponding, circumferentially aligned, third diagonal portion 328. In contrast, the first diagonal portion 324 can optionally be angled with respect to any of the corresponding second 326 and third 328 diagonal portions, extending along a length L3 as indicated in FIG. 3A. The lengths L1 of a second 326 diagonal portion can be substantially equal, in some examples, to the length L2 of a third diagonal portion 328 which is circumferentially aligned therewith, while the length L3 of a first diagonal portion 324 disposed therebetween, can be longer than any of the length L2 or the length L3 of the individual second 326 or the third 328 diagonal portion, respectively.

In some examples, the lengths L1 of all individual second diagonal portions 326, which can optionally be arranged in a zig-zagging pattern around the circumference of the frame, are equal to each other. In some examples, the lengths L2 of all individual third diagonal portions 328, which can optionally be arranged in a zig-zagging pattern around the circumference of the frame, are equal to each other. In some examples, the lengths L3 of all first diagonal portions 324, which can optionally be arranged in a zig-zagging pattern around the circumference of the frame, are equal to each other. While the example illustrated in FIGS. 3A-3B includes a plurality of individual continuously arranged second diagonal portions 326 having identical lengths L1, a plurality of individual continuously arranged third diagonal portions 328 having identical lengths L2 shown to be equal to L1, and a plurality of individual continuously arranged first diagonal portions 324 having identical lengths L3, it is to be understood that this is shown by way of illustration and not limitation, and that in some examples, two or more second diagonal portions 326 can optionally have different lengths, two or more third diagonal portions 328 can optionally have different lengths, and/or two or more first diagonal portions 324. Similarly, in some examples, a second diagonal portion 326 can optionally have a length L1 that is different from a length L2 of a third diagonal portion 328 which is circumferentially aligned therewith.

As illustrated in FIGS. 3A and 3B, consecutive second diagonal portions 326 can optionally together form a zig-zagged pattern around the circumference of the frame 104, wherein the inflow stitch points 352 and first intermediate stitch points 356 serve as distal and proximal vertices, respectively, of the zig-zagged pattern formed by the second diagonal portions 326. Optionally, consecutive third diagonal portions 328 can together form a zig-zagged pattern around the circumference of the frame 104, wherein the second intermediate stitch points 358 and outflow stitch points 354 serve as distal and proximal vertices, respectively, of the zig-zagged pattern formed by the third diagonal portions 328. The zig-zagged pattern formed by the second diagonal portions 326 can optionally be parallel to the zig-zagged pattern formed by the third diagonal portions 328. Consecutive first diagonal portions 324 can optionally together form a zig-zagged pattern around the circumference of the frame 104, wherein the inflow stitch points 352 and outflow stitch points 354 serve as distal and proximal vertices, respectively, of the zig-zagged pattern formed by the first diagonal portions 324.

In the example illustrated in FIGS. 3A and 3B, at least four outer portions 322 proximally extend from each inflow stitch point 352, and at least four outer portions 322 distally extend from each outflow stitch point 354. For example, with respect to FIG. 3B, two first diagonal portions 324a and 324b, and two second diagonal portions 326a and 326b, are shown to extend proximally from inflow stitch point 352a. Similarly, two first diagonal portions 324b and 324c, and two third diagonal portions 328b and 328c, are shown to extend distally from outflow stitch point 354b.

As mentioned, any outer skirt 300 disclosed herein, including exemplary outer skirt 300^{a}, can optionally be coupled to a frame of any prosthetic valve or other prosthetic device. When used in combination with the frame 104 of the mechanically expandable prosthetic valve described above with respect to FIGS. 1A-1C, and as shown for example in FIGS. 3A-3B, the outer skirt 300^{a} can optionally be assembled on frame 104 such that the inflow stitch points 352 are aligned with the inflow apices 114, and the outflow stitch points 352 are aligned with the post intermediate junctions 184. In some examples, the first intermediate stitch points 356 can optionally be aligned with the post inflow ends 180. In some examples, the second intermediate stitch points 358 can optionally be aligned with the lower post junctions 164.

A frame 104 of the design shown in FIGS. 1A-IC can optionally have relatively large spaces defines by relatively large cells 136, and outer portion 322 of multi-yarn threads(s) 318 arranged diagonally across such cells 136, such as following the pattern shown in FIGS. 3A-3B with a plurality of outer portions extending diagonally between vertical posts 122 of the frame 104, can advantageously provide adequate PVL sealing when implanted, while allowing for a smaller crimped profile during delivery to the site of implantation. For example, the outer portions 322 shown for outer skirt 300^{a} are designed to diagonally extend when the valve is in the expanded configuration, so as to provide PVL sealing across the entire circumference of the valve (as shown in FIG. 3B), yet when the valve is in a compressed or crimped configuration (not illustrated explicitly), the outer portions 322 can be arranged in a more vertical or axial orientation, allowing them to come closer to each other while preserving a relatively modest crimped profile. This is in contrast to alternative arrangements in which PVL sealing of the outer skirt can be achieved by a puffy pattern disposed over all, or at least most, of the outer surface of the skirt, in which case the outer skirt might increase the crimped profile of the valve.

FIGS. 4A-4B illustrate another implementation of outer skirt 300^{a} shown in a flattened configuration, in which some or all of the outer portions 322, including any of the first 324, second 326, and/or third 326 diagonal portions, can optionally further include one or more additional stitch point 350. In the illustrated example, the first diagonal portion 324 is shown to include an additional stitch point 350 between the inflow 352 and outflow 354 stitch points, such that the first diagonal portion 324 comprises two inner outer portions 322: an outer portion extending between the inflow stitch point 352 and the additional stitch point 350, and another outer portion extending the additional stitch point 350 and the outflow stitch point 354. The second diagonal portions 326 and third diagonal portions 328 are similarly shown, in the illustrated example, to include additional stitch points, dividing each into two (or more) shorter outer portions 322.

While additional stitch points 350 are illustrated for all of the first 322, second 326 and third 328 diagonal portions, it is to be understood that such a combination is shown by illustration and not limitation, and that any other combination is contemplated. For example, the first diagonal portion 324 can optionally include additional stitch points 350 between the inflow 352 and outflow 354 stitch points, while only one or none of the second 326 and third 328 diagonal lines include additional stitch points 350, compared to their arrangement in FIGS. 3A-3B for example. While a single additional stitch point 350 is shown for each of the first 322, second 326 and third 328 diagonal portions, it is to be understood that this is shown by illustration and not limitation, and that any of the outer portions 322, such as any of the first 322, second 326 and/or third 328 diagonal portions, can optionally include no additional stitch points, or more than one additional stitch point.

In some cases, it may be desirable to space away between subsequent stitch points 350 to allow for a longer slack of the multi-yarn thread 318 to freely extend over and radially outward to the base layer 302, since the yarns 370 of the multi-yarn thread 318 converge toward each other to a compacted, thinner configuration of the thread 318 at, as well as in the immediate vicinity of, the stitch point 350. Leaving a greater length of the thread 318 unattached via a stitch point 350 allows a greater portion of the thread 318 to obtain a “puffy” configuration, exhibiting a greater thickness, such as its free thickness T2, also termed herein a first thickness, along a longer portion, thereby potentially improving PVL sealing around the base layer 302 with reduced amount of material. However, on some occasions, having a loose slack which is too long may cause it to entangle with other components of the prosthetic valve or the delivery apparatus, during handling, preparation, and/or delivery of the valve. Thus, a balance should be sought for an outer portion 322 extending between two adjacent stitch points 350, to be long enough to allow for a greater slack of material which is free to assume its free-state thickness (i.e., its first thickness) T2, but not too long to reduce the risk of entanglement. For this reason, some patterns of the outer skirt 300 can include outer pattens with and without additional internal stitch points.

For example, a length threshold can be defined, below which no additional stitch point should be added to allow for a greater length material having a free-state thickness T2, and above which at least one additional stitch point 350 should be added to prevent or reduce the likelihood of entanglement. In some examples, second diagonal portions 326 do not include additional stitch points between the inflow 352 and first intermediate 356 stitch points, and third diagonal portions 328 do not include additional stitch points between the outflow 354 and second intermediate 358 stitch points, while the first diagonal portions 324, which are longer and may exceed such a length threshold, do include at least one additional stitch point 350 between the inflow 352 and outflow 354 stitch points.

FIG. 5A shows an exemplary skirt 300^{b} disposed over a frame 104 of valve 100^{b} in a flattened configuration, and FIG. 5B shows a view in perspective of the valve 100^{b} with outer skirt 300^{b} of FIG. 5A. Outer skirt 300^{b} is an exemplary implementation of outer skirt 300, and thus includes all of the features described for outer skirt 300 throughout the current disclosure. Specifically, outer skirt 300^{b} can be similar to any example of outer skirt 300^{a} described above, except that the outer portions 322 further include fourth diagonal portions 330 and fifth diagonal portions 332. The stitch points 350 can optionally further include third intermediate stitch points 360 and fourth intermediate stitch points 362.

Third intermediate stitch points 360 are defined on the second diagonal portions 326, such that each third intermediate stitch point 360 is positioned between the inflow 352 and first intermediate 356 stitch points of the respective second diagonal portion 326. Fourth intermediate stitch points 362 are defined on the third diagonal portions 328, such that each fourth intermediate stitch point 362 is positioned between the outflow 354 and second intermediate 358 stitch points of the respective third diagonal portion 328.

Each fourth diagonal portion 330 extends between an outflow stitch point 354 and a third intermediate stitch points 360. Since third intermediate stitch points 360 are circumferentially offset from both the outflow stitch points 354 and the inflow stitch points 352, the fourth diagonal portions 330 are angled with respect to the longitudinal axis C, at an angle that can be different than that of any of the first 324, second 326 and third 328 diagonal portions.

Each fifth diagonal portion 332 extends between an inflow stitch point 352 and a fourth intermediate stitch points 362. Since fourth intermediate stitch points 362 are circumferentially offset from both the outflow stitch points 354 and the inflow stitch points 352, the fifth diagonal portions 332 are angled with respect to the longitudinal axis C, at an angle that can be different than that of any of the first 324, second 326 and third 328 diagonal portions.

In some examples, at least six outer portions 322 proximally extend from each inflow stitch point 352, and at least six outer portions 322 distally extend from each outflow stitch point 354. For example, with respect to FIG. 5B, two first diagonal portions 324a and 324b, two second diagonal portions 326a and 326b, and two fifth diagonal portions 332 and 332b, are shown to extend proximally from inflow stitch point 352a. Similarly, two first diagonal portions 324b and 324c, two third diagonal portions 328b and 328c, and two fourth diagonal portions 330b and 330c, are shown to extend distally from outflow stitch point 354b.

In some examples, an outer section 322 can optionally be a vertical outer section, extending axially parallel to the longitudinal axis C. In the example illustrated in FIGS. 5A-5B, the outer sections 322 further include first vertical portions 334 and second vertical portions 336. Each first vertical portions 334 extends between an inflow stitch point 352 and a second intermediate stitch point 358. Each second vertical portions 336 extends between an outflow stitch point 354 and a first intermediate stitch point 356. The first vertical portions 334 and the second vertical portions 336 extend axially, parallel to each other and to the longitudinal axis C.

When used in combination with the frame 104 of the mechanically expandable prosthetic valve described above with respect to FIGS. 1A-1C, and as shown for example in FIGS. 5A-5B, the outer skirt 300^{b} can optionally be assembled on frame 104 such that the first vertical portions 334 are aligned with lower post members 164. In some examples, the second vertical portions 336 can be aligned with support posts 124.

In some examples, at least seven outer portions 322 proximally extend from each inflow stitch point 352, and at least seven outer portions 322 distally extend from each outflow stitch point 354. For example, with respect to FIG. 5B, in addition to the six diagonal outer portions 322 described above, a first vertical portion 334 extends proximally from inflow stitch point 352a. Similarly, in addition to the six diagonal outer portions 322 described above, a second vertical portion 336 extend proximally from outflow stitch point 354b.

While outer skirt 300^{b} is illustrated to include both the first vertical portions 334 and the second vertical portions 336, it is to be understood that in some examples, first vertical portions 334 can optionally be included without second vertical portions, and that in some examples, second vertical portions 336 can optionally be included without first vertical portions.

While outer skirt 300^{b} is shown to combine fourth 330 and fifth 332 diagonal portions with first 334 and second 336 vertical portions, it is to be understood that such combinations are not mandatory, and that outer portions 322 of any outer skirt 300 can optionally include fourth 330 and/or fifth 332 diagonal portions without inclusion of vertical portions. Similarly, any outer skirt 300 can optionally include first 334 and/or second 336 vertical portions without the fourth and fifth diagonal portions. For example, an outer skirt that includes first 324, second 326 and third 328 diagonal section, such as skirt 300^{a} illustrated in any of FIGS. 3A-4, can optionally further include first 334 and second 336 vertical portions. In such cases, at least five outer portions 322 can optionally proximally extend from each inflow stitch point 352 by further including a first vertical portion 334 extending proximally therefrom, and at least five outer portions 322 can optionally distally extend from each outflow stitch point 354 by further including a second vertical portion 334 extending distally therefrom.

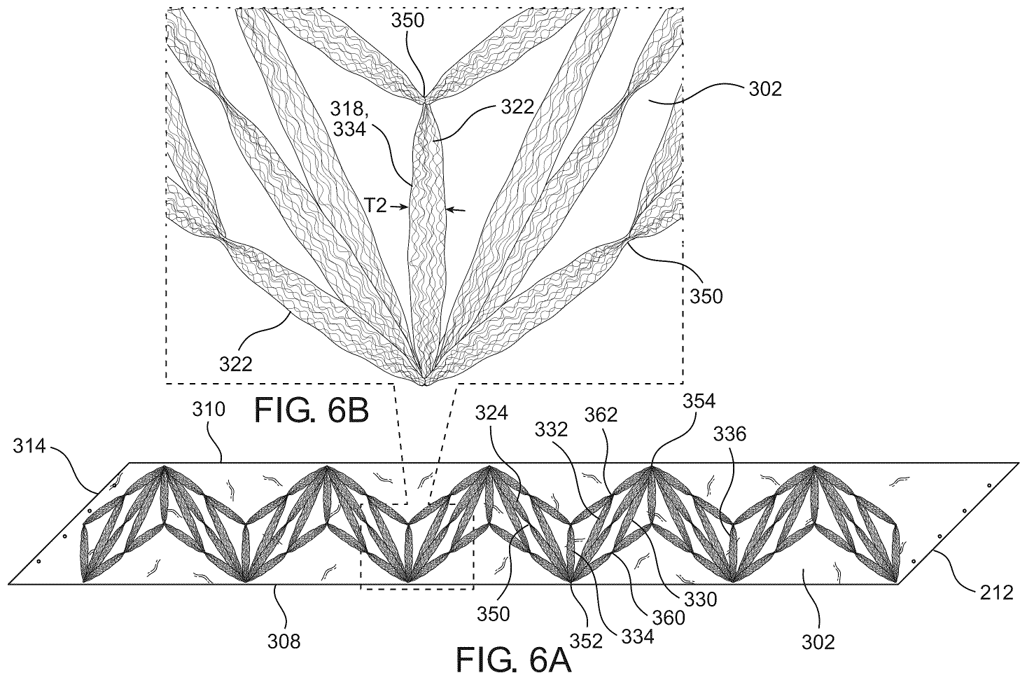

FIG. 6A is a schematic of another implementation of outer skirt 300^{b} shown in a flattened configuration, in which some or all of the outer portions 322, including any of the first 324, second 326, third 326, fourth 330 and/or fifth 332 diagonal portions, as well as any of the first 334 and/or second 336 vertical portions, can optionally further include one or more additional stitch point 350, in a manner similar to any of the examples described above with respect to FIGS. 4A-4B. FIG. 6B shows an enlarged section of FIG. 6A in the vicinity of an inflow stitch point 352.

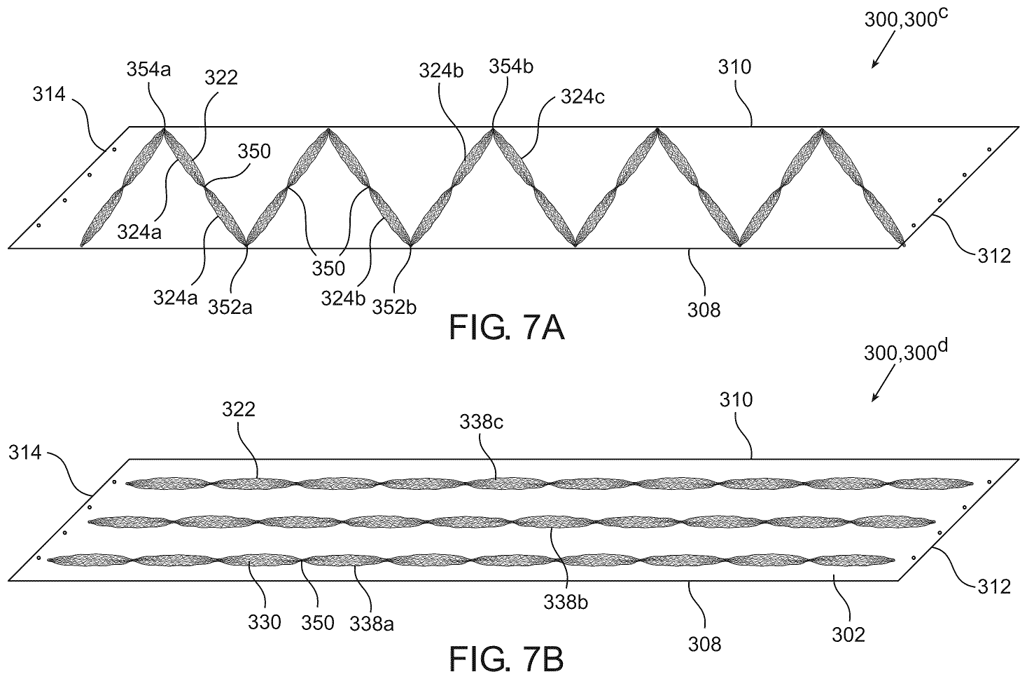

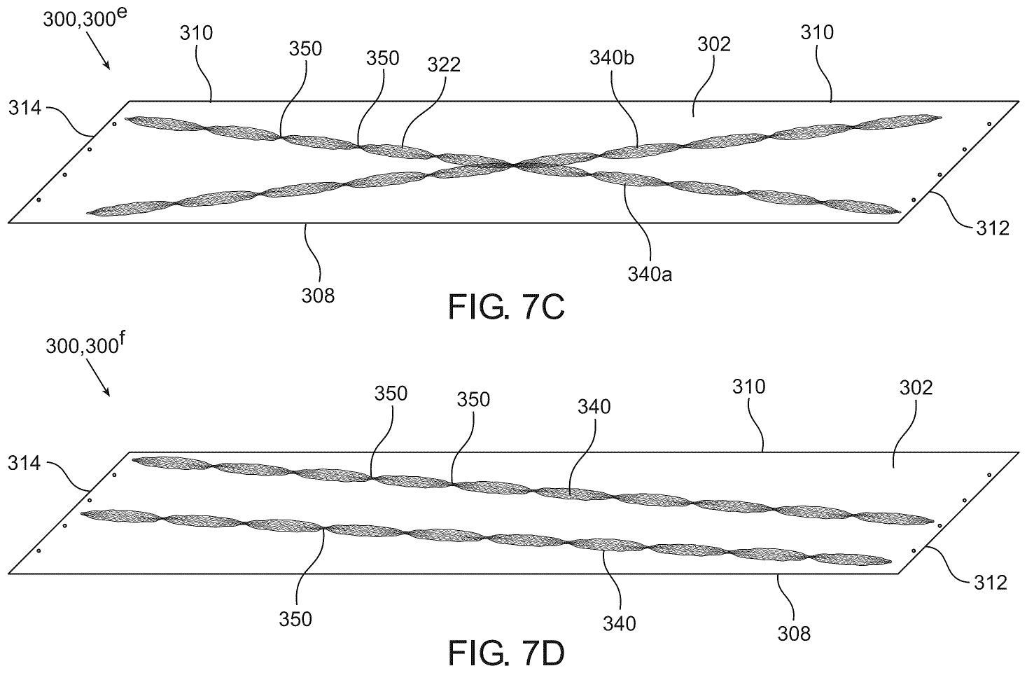

FIGS. 7A-7D show additional exemplary outer skirt 300 illustrated in flattened configurations thereof, each of which is an exemplary implementation of outer skirt 300, and thus includes all of the features described for outer skirt 300 throughout the current disclosure, except for the pattern of the multi-yarn thread 320 over the base layer 302. FIG. 7A shows an exemplary skirt 300€ that includes first diagonal portions 324 extending between inflow stitch points 352 and outflow stitch points 354, which together form a zig-zagged pattern around the circumference of the base layer 302. This can resemble the pattern described above with respect to outer skirt 300^{a}, omitting the second and third diagonal layers. While each first diagonal portion 324 is shown to include an additional stitch point 350 between the respective inflow 352 and outflow 354 stitch points, it is to be understood that this is shown by illustration and not limitation, and that any of the first diagonal portions 324 can optionally be devoid of any additional stitch point, or optionally include more than a single stitch point 350.

FIG. 7B shows an exemplary skirt 300^{d} that includes outer portions 322 in the form of circumferential portions 338, extending around the circumference of the base layer 302, between the first 312 and second 314 side edge portions, optionally parallel to the inflow edge portion 308. While three circumferential portions 338 are illustrated, any other number, including one, two, or more than three circumferential portions, is contemplated. Any circumferential portion 338 can optionally include any desired number of stitch points 350 along a length thereof.

FIG. 7C shows an exemplary skirt 300^{e} that includes at least two outer portions 322 in the form of angled circumferential portions 340 that cross each other. Each angled circumferential portion 340 can optionally extend around the circumference of the base layer 302, such as between the first 312 and second 314 side edge portions, in an angled orientation with respect to central axis C, such as between the inflow 308 and outflow 310 edge portions. The two angled circumferential portion 340 are angled at opposite orientations relative to each other, resulting in one the angled circumferential portion 340 crossing each other over the outer surface 380 of base layer 302, together forming an X-shaped pattern. Several X-shaped patterns can optionally be similarly arranged around the circumference of the base layer 302. Any angled circumferential portion 340 can optionally include any desired number of stitch points 350 along a length thereof.

FIG. 7D shows an exemplary skirt 300^{f} that illustrated to include two outer portions 322 in the form of angled circumferential portions 340 extending parallel to each other. While two angled circumferential portions 340 are illustrated for skirt 300^{f}, any other number is contemplated, such as a single angled circumferential portion 340 or more than two angled circumferential portions 340. Any angled circumferential portion 340 can include any desired number of stitch points 350 along a length thereof.

According to some examples, the multi-yarn thread 318 includes yarns 360 which are interweaved by braiding, twisting, plying, and the like. Optionally, the yarns 360 are interweaved by braiding. Thus, according to some examples of the present method, the multi-yarn thread 318 is a braided thread.

However, it is to be understood that while a braided configuration of the multi-yarn thread 318 is described for some examples above (e.g., with respect to FIGS. 3A-7D), other threads, which are not necessarily braided, can be used in a similar manner to form the patterns according to any of the examples described with respect to outer skirts 300 shown, e.g., in FIGS. 3A-7D above. For example, a non-braided thread that has a free-state thickness T2 (i.e., when the thread is not tensioned and not compressed, also termed first thickness T2) which is greater than the thickness T1 of base layer 302, can optionally define outer portions that extend over the outer surface 380 of the base layer 302 so as to form any pattern described hereinabove with respect to FIGS. 3A-7D. In another example, a twisted or ply thread may similarly define the same thickness and/or define outer portions that extend over the outer surface 380 of the base layer 302.

While thread 318 is described above with respect to FIGS. 3A-7D to define outer portions 322 between stitch points 350 at which it is stitched to the base layer 302, it is to be understood that multi-yarn threads 318 can optionally be coupled to the base layer 302 by other coupling means instead of, or in addition to, stitching. Thus, the term “stitch point” with respect to attachment points of the thread 318 to the base layer 302 is not meant to be limiting, and that any reference to a “stitch point 350” with respect to FIGS. 3A-7D, as well as any type of stitch points, such as inflow stitch point 352, outflow stitch point 354, and the like, can be similarly referred to as a “coupling point 350”, which can include inflow coupling points 352, outflow coupling point 354, and the like.

When the thread defining the outer portions 322 is a multi-yarn thread 318, which comprises a plurality of yarns 370, and wherein each yarn 370 can be a multi-filament yarns. A multi-yarn thread 318 can optionally be formed such that in a free state thereof, the plurality of yarns 370 are fuzzy, wavy, and/or spaced away from each other, result in a “puffy” of expanded thickness of the thread 318 that can reach a thickness of about T2. A free state of the multi-yarn thread 318 is defined as a state in which the yarns are not tensioned between adjacent stitch points 350, and during which multi-yarn thread 318 is not compressed, such as between the base layer 302 and an outer surface, which can optionally be a component of the delivery apparatus 200 (e.g., capsule 210 or outer shaft 208) or an anatomical wall, such as the native annulus against which the skirt 300 can be pressed when the valve 100 is implanted. It is to be understood that the shape and spacing between the yarns 370 in the free state can very, such that T2 can optionally be an average value of the thickness of a portion of the multi-yarn thread 318 in the free state, or a minimal value of a portion of the multi-yarn thread 318 in the free state.

In some examples, multi-yarn thread 318 comprise at least 4 yarns 370. In some examples, the multi-yarn thread 318 comprises between 4 and 16 yarns 370. In some examples, each yarn 370 of the multi-yarn thread 318 is a bulk textured yarn which has a weight between 10 Denier to 150 Denier. Denier (D) is a unit of measure for linear mass of each fiber comprising the mass in grams per 9000 meters of the fiber. In some examples, each yarn 370 has a weight between 12 D to 140 D. In some examples, each yarn 370 has a weight between 14 D to 130 D. In some examples, each yarn 370 has a weight between 15 D to 120 D. In some examples, each yarn 370 has a weight between 16 D to 110 D. In some examples, each yarn 370 has a weight between 17 D to 100 D. In some examples, each yarn 370 has a weight between 18 D to about 90 D. In some examples, each yarn 370 has a weight between 20 D to 80 D.

In some examples, the multi-yarn thread 318 has a filament count between 5 to 150 per yarn. In some examples, the multi-yarn thread 318 has a filament count between 10 to 48 per yarn. In some examples, the multi-yarn thread 318 has a filament count of at least 5. In some examples, the multi-yarn thread 318 has a filament count of, at least 6 filaments per yarn., In some examples, the multi-yarn thread 318 has a filament count of at least 7 filaments per yarn., In some examples, the multi-yarn thread 318 has a filament count of at least 8 filaments per yarn. In some examples, the multi-yarn thread 318 has a filament count of at least 9 filaments per yarn. In some examples, the multi-yarn thread 318 has a filament count of or at least 10 filaments per yarn.

In some examples, the multi-yarn thread 318 has a density between 5 to 50 Picks Per Inch (PPI), which is the number of interlacing points per inch of thread. In some examples, the multi-yarn thread 318 has a density between 10 to 20 PPI.

In some examples, the multi-yarn thread 318 can optionally comprise a plurality of nanofilament yarns. In some examples, the multi-yarn thread 138 can optionally comprise a plurality of multi-nanofilament yarns. In some examples, the multi-yarn thread 318 can optionally be braided with a core of monofilament yarns.

In some examples, the multi-yarn thread 318 can optionally be braided with a core of monofilament such that the monofilament is made of low-melt biocompatible polymer suitable for long-term implant application. This monofilament can optionally have diameter ranging from 10 micron to 250 micron.

In some examples, the intent of the monofilament is to provide reinforcement to the core of the multi-thread yarn thread 318 such that it can be thermoformed to create outward protruding arcs away from the central longitudinal axis C. Also, in some examples, with the core of the low-melt polymer, the fabric can optionally be passed over hot plate or roller so the multi-yarn thread 318 on the back of the fabric will be softened or melted to lock the multifilament filaments in place. In some examples, the low-melt polymer can optionally have melting temperature in range of 20° C.-130° C. lower than a base polymer that makes the multi-yarn thread 318. In some examples, the base polymer such as Polyethylene Terephthalate (PET) or polyester that can make up the multi-yarn thread has melting temperature of 260° C., and the low-melt polymer can optionally have melting temperature from 240° C. to as low as 130° C.

In some examples, the monofilament can optionally have diameter in the range of 10 micron to 250 micron. In some examples, each monofilament can optionally have diameter in the range of 10 micron to 250 micron.

In some examples, the monofilament yarns comprise low-melt biocompatible polymer. In some examples, the monofilament yarns comprise at least one low-melt polymer. In some examples, the low-melt polymer has a melting temperature in the range of 130° C. to 240° C.

In some examples, the monofilament yarns comprise biocompatible polymer.

Various stitching techniques can be employed to couple the multi-yarn thread 318 to base layer 302. For example, the multi-yarn thread can optionally be loose or unattached to the base layer 302 along outer portions 322 extending between subsequent stitch points 350, wherein the multi-yarn thread 320 can optionally be stitched to the base layer 302 at the stitch points 350. Stitching the multi-yarn thread 318 to the base layer 302 can optionally include, in some examples, passing the thread 318 through the thickness of the base layer 302. In some examples, the thread 318 can optionally be stitched through the base layer 302 in an in-and-out passing at the stitch point 350, meaning that the thread 318 can be passed, such as by a needle, from one side of base layer 302 to the opposite side, and then passed back to the first side, such as by passing the needle a second time in the opposite direction to form a hole in close proximity to the first hole. In such examples, two closely-positioned holes through which the suture 318 is passed in a first direction and back in an opposite direction through the base layer 302, can together define the region of a stitch point 350.

In some examples, stitching the multi-yarn thread 320 to the base layer 302 can be performed manually, such as with a hand-held needle. However, forming a desired pattern can be a tedious and time-consuming procedure when performed manually, and therefor also subject to human errors. It may be desired to reduce the time it takes to form the desired pattern of the thread 318 over the base layer 302, in a manner that can make the suturing or stitching process easier and quicker, and lead to more accurate assembly procedure with less errors.

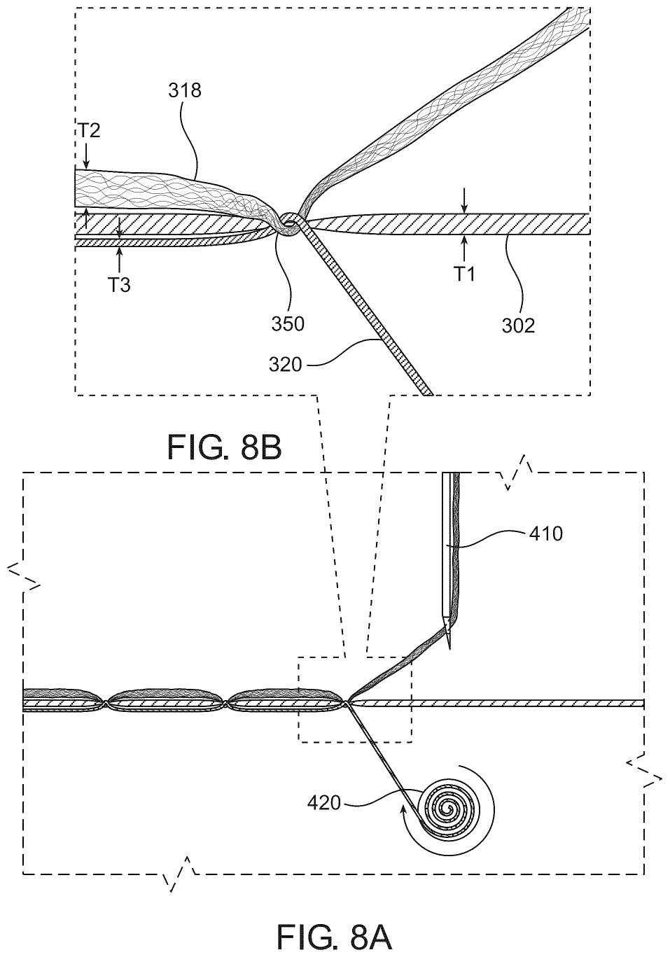

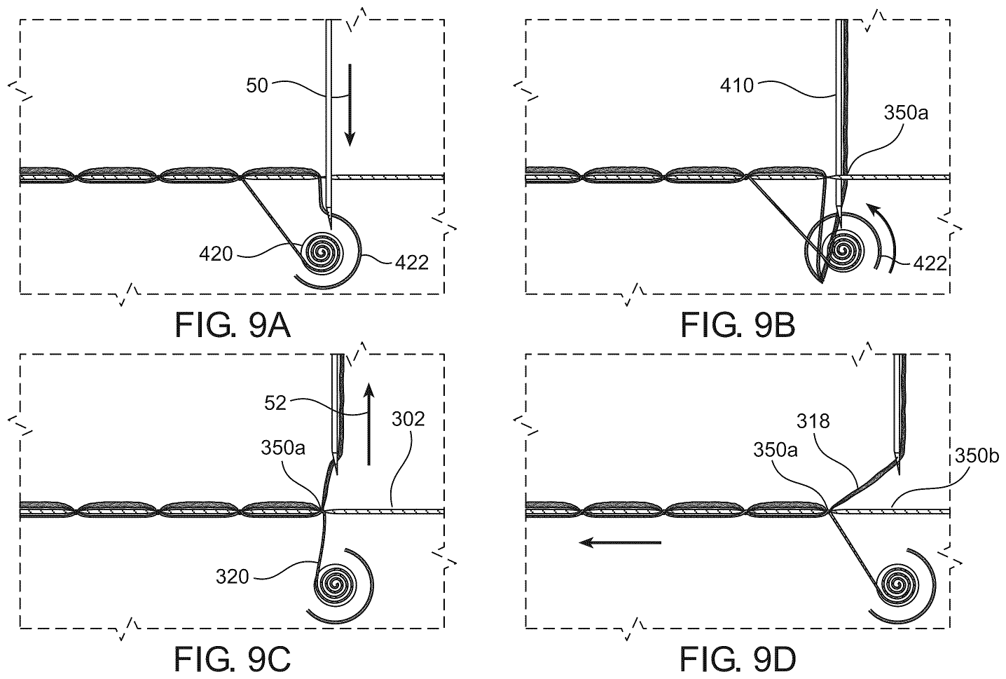

In some examples, embroidery stitching is utilized for forming the desired pattern of multi-yarn thread(s) 318 over base layer 302 of outer skirt 300. FIGS. 8A-9D illustrate an exemplary lock-stitch embroidery patterning method for forming the outer skirt 300. In some examples, the lock-stitch embroidery patterning method disclosed herein is utilized for attaching the multi-yarn thread(s) 318 to the base layer 302, forming outer portions 322 of the multi-yarn thread 318 extending over the outer surface 380 of the base layer 304. Any one of the patterns described above with respect to FIGS. 3A-7D can optionally be formed by the method disclosed herein.

In some examples, there is provided an embroidery patterning method for forming and assembling an outer skirt 300 of a prosthetic valve 100, comprising some or all of the following steps:
(a) providing an automated sewing machine 400, which comprises an embroidery needle 410 and an embroidery bobbin 420 assembled to a rotary hook 422;
(b) providing a backing thread 320, wherein the backing thread 320 is coiled around the embroidery bobbin 420;
(c) providing a multi-yarn thread 318, wherein the multi-yarn thread 318 is held by the embroidery needle 410;
(d) spreading a base layer 302 in the automated sewing machine 400 such that its inner surface 382 is facing the embroidery bobbin 420 and its outer surface 380 is facing the embroidery needle 410;
(e) pulling a section of the backing thread 320 from the embroidery bobbin 420 toward the inner surface 382 of the base layer 302 at a first stitch point 350a;
(f) passing the needle 410 with the multi-yarn thread 318 through the base layer 300 at the first stitch point 350a;
(g) lacing the multi-yarn thread 318 around the backing thread 320 using the rotary hook 422;
(h) withdrawing the embroidery needle 410 from the base layer 302, thereby forming a stitch of the multi-yarn thread 318 and the backing thread 320 at the first stitch point 350;
(i) moving the base layer 302 to position a subsequent stitch point 350b between the embroidery needle 410 and the embroidery bobbin 420;
(j) repeating steps (e) to (h) to form an additional stitch at the subsequent stitch point 350b, thereby forming an outer portion 322 of the multi-yarn thread 318 extending between the first stitch point 350a and the subsequent stitch point 350b;
(k) optionally repeating, one or more times, step (i) to position another subsequent stitch point 350 between the needle 410 and the bobbing 420, and steps (e) to (h) to form another stitch at the subsequent stitch point 350, thereby forming at least one additional successive outer portion 322.
(l) attaching the base layer 302 to a frame 104 of a prosthetic valve 104, such that the inner surface 382 of the base layer 302 is facing the frame 104.

The base layer 302 provided in step (d) above can optionally be cut to any desired shape, such as having angled first 312 and second 314 side edge portions. The outflow edge portion 310 of the base layer 302 can optionally be parallel to the inflow edge portion 310 as illustrated, or can have any other shape, such as including extensions that can optionally follow the shapes of struts of specific frame designs, as mentioned above.

The method can optionally be utilized to form one or more outer portions 322 between subsequent stitch points 350 by repeating steps (e) to (i) as many times as required, to achieve a desired pattern of the multi-yarn thread(s) 318 over base layer 302. Moreover, more than one multi-yarn thread 318 can optionally be stitched according to the above method, so as to form various outer pattern 322 from more than one multi-yarn thread 318, together resulting in a desired pattern over a single base layer 302.

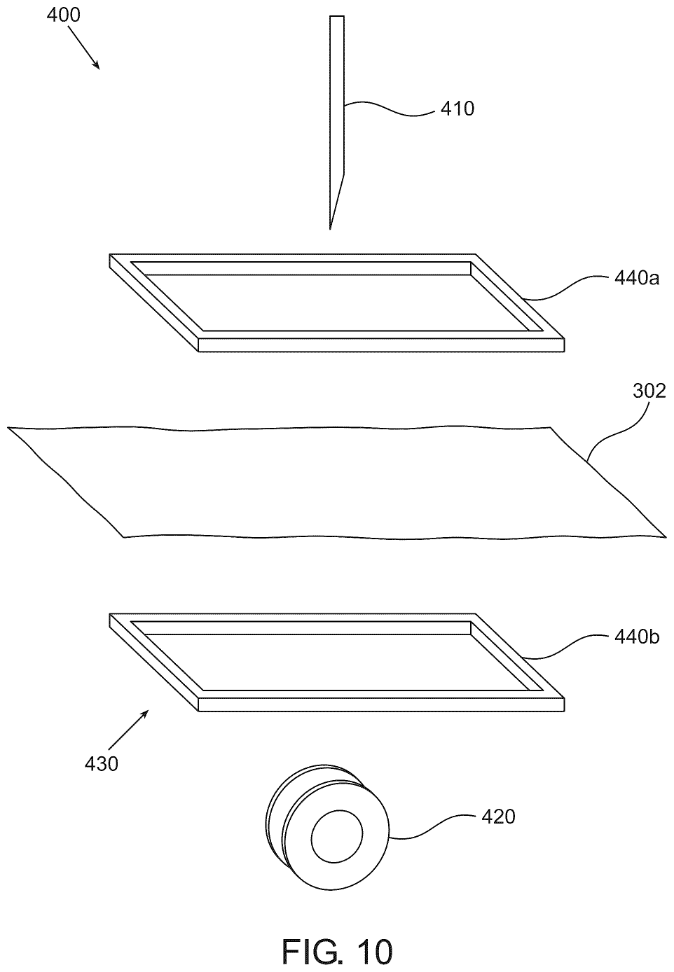

FIG. 8A shows a lock-stitch formed at a stitch point 350 upon lacing the multi-yarn thread 318 on the backing thread 320, according to some examples. FIG. 8B shows an enlarged section of FIG. 8A in the vicinity of the stitch point 350. FIGS. 9A-9D illustrate some steps of the present method, as discussed in further detail below. FIG. 10 shows some components of an exemplary automated sewing machine 400 that can be employed in the present embroidery method for production of an outer skirt 300 with a patterned multi-yarn thread 318. The components shown in FIG. 10 can optionally be of an automated sewing machine 400 which can be provided in step (a) of the method described above.

Specifically, it is highly advantageous and economical in the field of medical devices in general and prosthetic heart valves (such as prosthetic valve 100) in particular to incorporate automated machinery, replacing the slow, occasionally inaccurate and expensive human labor, which constitutes the typical manual sewing procedures conventionally employed in this field. Suturing of soft components of prosthetic valve devices and/or other implant devices, such as those described above, can be performed in various ways. For example, certain manual procedures for suturing components of prosthetic implant devices can be implemented, in which an operator utilizes both hands for holding, securing, and/or suturing the implant device. For example, a human operator may stitch suture lines over one or more layers of skirts.

Examples disclosed herein may provide improved quality control to processes which are conducted by human labor. For example, suturing an implant device or heart valve can require suture accuracy within a millimeter, half a millimeter, or less, but a suture location may be easily missed between ribs or threads, especially when implementing dual-handheld suturing procedures. Examples of the present disclosure can facilitate improved precision through the use of automated machinery.

Positional accuracy may be improved with respect to examples of the present disclosure through the use of systems incorporating one or more cameras, hooks, automated fixtures, monitors, etc., and/or a combination of more than one of these. Such systems can be used to position a target stitch in a desirable position over the base layer 302 with a relatively high degree of accuracy and repeatability.

Importantly, while regular suturing is not compatible with automation in such small systems, such as outer skirts, embroidery machinery was found to produce desirable results including accurate lock stitching in a fast rate.

Thus, according to some examples, the present method comprises step (a) of providing an automated sewing machine 400. In some examples, the sewing machine 400 comprises an embroidery needle 410 and an embroidery bobbin 420. In some examples, the embroidery bobbin 420 is assembled to a rotary hook 422. FIG. 10 illustrates an exemplary embroidery needle 410 and an exemplary embroidery bobbin 420.

According to some examples of the present method, the sewing machine 400 further comprises a tensioning fixture 430. In some examples, a tensioning fixture 430, which is not necessarily part of the sewing machine 400, is separately provided to allow spreading and tensioning of the base layer 302. In some examples, the tensioning fixture 430 comprises a first fixture portion 440a and a second fixture portion 440b, which together are arranged to affix sheets, such as base layer 302, at a tensioned state. The first fixture portion 440a and the second fixture portion 440b can optionally be together arranged to affix sheets, such as base layer 302, at a spread state. In some examples, each one of the first portion 440a and the second fixture portion 440b can optionally be in the form of a framed screen and include a matching alignment template, such that a base layer 302 positioned between the first portion 440a and the second fixture portion 440b may be affixed at a spread tensioned state.

In some examples, the method further comprises step (b) of providing a backing thread 320 defining a thickness T3, also termed herein a second thickness. The backing thread 320 is described in detail in the present disclosure both individually and as compared to the multi-yarn thread 318. In some examples of the method, the backing thread 320 can optionally be coiled around the embroidery bobbin 420.

In some examples, T3 remains uniform along various portions of the backing thread 320, including portions thereof extending between stitch points 350 and/or portions thereof disposed in the stitch points 350. Stated otherwise, the backing thread 320 can optionally be less compressible than the multi-yarn thread 318.

In some examples, the backing thread 320 comprises 1 to 6 yarns. In some examples, the backing thread 320 comprises 1 to 5 yarns. In some examples, the backing thread 320 comprises 1 to 4 yarns. In some examples, the backing thread 320 comprises 1 to 3 yarns. In some examples, the backing thread 320 comprises 1 or 2 yarns. In some examples, the backing thread 320 comprises single yarn.

In some examples, the backing thread 320 comprises at least one flat yarn and/or at least one twisted yarn. In some examples, the backing thread 320 comprises a single flat yarn or a twisted yarn. It is to be understood that the term “flat yarn” refers to a non-twisted yarn. As can further be appreciated by the person having ordinary skill in the art, the twisting degree of twisted yarns is typically defined by twists per inch (TPI). In some examples, the backing thread 320 comprises a single flat yarn. In some examples, the backing thread 320 comprises a single twisted yarn. In some examples, the twisted yarn has between 2 to 16 TPI.

In some examples, the backing thread 320 comprises at least one yarn weight that can optionally be defined as between 5 D to 75 D. In some examples, the backing thread 320 comprises at least one yarn which has a weight between 7 D to 60 D. In some examples, the backing thread 320 comprises at least one yarn which has a weight between 8 D to 55 D. In some examples, the backing thread 320 comprises at least one yarn which has a weight between 9 D to 50 D. In some examples, the backing thread 320 comprises at least one yarn which has a weight between 10 D to 40 D.

In some examples, the backing thread 320 comprises at least one yarn including a polyester, a polyolefin, polytetrafluoroethylene (PTFE), silk, and a thermoplastic polyurethane (TPU): ultra-high-molecular-weight polyethylene (UHMWPE) hybrid or a combination thereof. In some examples, the backing thread 320 comprises at least one yarn comprising polyester. In some examples, the backing thread 320 comprises at least one yarn comprising a polyolefin. In some examples, the polyolefin comprises polyethylene or a copolymer thereof. In some examples, the polyolefin comprises polyethylene. According to some examples, the polyethylene comprises ultra-high-molecular-weight polyethylene (UHMWPE). In some examples, the backing thread 320 comprises at least one yarn comprising silk. In some examples, the backing thread 320 comprises at least one yarn comprising PTFE. In some examples, the backing thread 320 comprises at least one yarn comprising a TPU: UHMWPE hybrid. In some examples, the backing thread 320 comprises at least one yarn comprising a biocompatible material. In some examples, the backing thread 320 comprises at least one yarn consisting of a biocompatible material(s).

In some examples, the backing thread 320 comprises at least one yarn made from low-melt polymer. In some examples, at least one yarn made from low-melt polymer enables the fabric can be post-processed for thermal treatment. With the thermal treatment, the post-processing temperature is elevated to softening temperature and close to melting temperature of the low-melt polymer, in some examples. This, in some examples, allows the backing material lock in place where the stitches are made. In some examples, the low-melt polymer has a melting temperature in the range of 130° C. to 240° C.

In some examples, the backing thread 320 comprises at least one implantable biocompatible metal or metal alloy wire. In some examples, the implantable biocompatible metal or metal alloy wire has wire diameter in the range of about 10 micron to about 100 micron. In some examples, the implantable biocompatible metal or metal alloy wire can optionally comprise an alloy. In some examples, the implantable biocompatible metal or metal alloy wire can optionally comprise a flexible alloy. In some examples, the implantable biocompatible metal or metal alloy wire can optionally comprise stainless steel, NiTi, gold, platinum or a combination thereof.

In some examples, the role of the metal wires would be to provide a low-profile backing thread as well in certain case provide radiopaque feature based upon the placement of the backing materials.

In some examples, the backing thread 320 comprises a bicomponent polymer. In some examples, the backing thread 320 is made of a bicomponent polymer. In some examples, the bicomponent polymer has core and sheath structure.

In some examples, the backing thread 320 is made of bicomponent polymer with core and sheath structure in the extruded filaments that makes the final yarn. Such bicomponent polymer filaments can have outer sheath made of low melting temperature polymer and the inner core can have a higher melting temperature.

In some examples, the outer sheath comprises at least one low melting temperature polymer. In some examples, the outer sheath comprises a low melting temperature polymer. In some examples, the outer sheath consists of a low melting temperature polymer. In some examples, the low-melt polymer has a melting temperature in the range of 130° C. to 240° C.

In some examples, the inner core comprises a higher melting temperature polymer. In some examples, the inner core comprises at least one higher melting temperature polymer. In some examples, the inner core consists of a higher melting temperature polymer. In some examples, the high-melt polymer has a melting temperature higher than that of the low-melting polymer. In some examples, the high-melt polymer has a melting temperature of at least 250° C.

In some examples, the core can optionally be PET having a melting temperature of 260° C. and the sheath can optionally be TPU (or other low-melt temperature polymer) with melting temperature in the range of 100° C.-200° C. suitable for long-term implant application.

In some examples, the bicomponent filament yarn can optionally have weight of 0.2 D to 10 D per filament. In some examples, the filament count in the yarn can optionally range from 1 to 68.

In some examples, the backing thread 320 is made of a coated or covered bicomponent polymer. In some examples, the backing thread 320 comprises a coated bicomponent polymer. In some examples, the backing thread 320 consists of a coated bicomponent polymer.

In some examples, the backing thread 320 made of a coated or covered bicomponent polymer comprises a core and a coating. In some examples, the core comprises the multifilament yarn. In some examples, the coating comprises a sheath. In some examples, the core multifilament yarn has a higher melting temperature than the outer sheath coating. In some examples, the core multifilament yarn has a melting temperature, which is at least 50° C. higher than the outer sheath coating. In some examples, the core multifilament yarn has a melting temperature, which is at least 100° C. higher than the outer sheath coating.

In some examples, core multifilament yarn comprises a polyester. In some examples, core multifilament yarn comprises PET. In some examples, the outer sheath coating comprises thermoplastic polyurethane (TPU), high density polyethylene (HDPE), a copolymer or combination thereof. In some examples, coating of the outer sheath may optionally be performed by one or more of known coating methods such as dip-coating, kiss-coating, spray coating, electrospinning etc. and/or combination of those but not limited to only the listed coating methods.

In some examples, the present method further comprises step (c) of providing a multi-yarn thread 318 which defined a thickness T2 as described above. In some examples, the multi-yarn thread 318 is held by the embroidery needle 410. As mentioned above, the multi-yarn thread can optionally include a plurality of yarns 370, each of which can optionally be a multi-filament yarn. The plurality of yarns 370 can optionally form a fuzzy texture of the multi-yarn thread 318, at least in a free state thereof. In some examples, the plurality of yarns 370 can optionally extend in a wavy pattern along the multi-yarn thread 318.

In some examples, T2 is at least 50% greater than T3. In some examples, T2 is at least 100% greater than T3. In some examples, T2 is at least 200% greater than T3. In some examples, T2 is at least 300% greater than T3. In some examples, T2 is at least 400% greater than T3. In some examples, T2 is at least 500% greater than T3. In some examples, T2 is at least 600% greater than T3. In some examples, T2 is at least 700% greater than T3. In some examples, T2 is at least 800% greater than T3. In some examples, T2 is at least 900% greater than T3. In some examples, T2 is at least 1000% greater than T3.

As illustrated, when the multi-yarn thread 318 is sewn to the base layer 302, it may have a minimal thickness at the stitch points 350, from which it gradually expands along portions thereof extending away from the stitch points 350. Depending on the le length of the outer portion 322 between two successive stitch point 350, the multi-yarn thread 318 can optionally assume a free-state thickness T2 at an intermediate point or region between the two subsequent successive stitch point 350.

In some examples, the free state thickness T2 is at least 50% greater than the minimal thickness of the multi-yarn thread 318 at the stitch points 350. In some examples, the free state thickness T2 is at least 100% greater than the minimal thickness of the multi-yarn thread 318 at the stitch points 350. In some examples, T2 is at least 200% greater than the minimal thickness of the multi-yarn thread 318 at the stitch points 350. In some examples, T2 is at least 300% greater than the minimal thickness of the multi-yarn thread 318 at the stitch points 350. In some examples, T2 is at least 400% greater than the minimal thickness of the multi-yarn thread 318 at the stitch points 350. In some examples, T2 is at least 500% greater than the minimal thickness of the multi-yarn thread 318 at the stitch points 350.

According to some examples, the multi-yarn thread 318 includes yarns 360, which are interweaved by braiding, twisting, plying, and the like. Optionally, the yarns 360, are interweaved by braiding. Thus, according to some examples, the multi-yarn thread 318 is a braided thread. In some examples, the multi-yarn thread 318 comprises between 3 to 25 yarns 370. In some examples, the multi-yarn thread 318 comprises between 4 to 16 yarns 370. In some examples, the multi-yarn thread 318 comprises at least four yarns 370. In some examples, the multi-yarn thread 318 comprises at least five yarns 370. In some examples, the multi-yarn thread 318 comprises no more than 30 yarns 370. In some examples, the multi-yarn thread 318 comprises no more than 25 yarns 370. In some examples, the multi-yarn thread 318 comprises no more than 20 yarns 370. In some examples, the multi-yarn thread 318 comprises no more than 16 yarns 370.

In some examples, the multi-yarn thread 318 comprises at least one yarn comprising a polyester or a copolymer thereof, thermoplastic polyurethane (TPU), a polyolefin or any combination thereof. In some examples, the multi-yarn thread 318 comprises at least one yarn comprising a polyester or a copolymer thereof. In some examples, the multi-yarn thread 318 comprises at least one yarn comprising a polyester. In some examples, the multi-yarn thread 318 comprises at least one yarn comprising a polyester copolymer. In some examples, the multi-yarn thread 318 comprises at least one yarn comprising TPU. In some examples, the multi-yarn thread 318 comprises at least one yarn comprising a polyolefin. In some examples, the polyolefin comprises polyethylene, polypropylene, polyvinylchloride (PVC) or any copolymer or combination thereof. In some examples, the polyolefin is selected from the group consisting of: polyethylene, polypropylene, polyvinylchloride (PVC) and any copolymer thereof. In some examples, the polyolefin comprises polyethylene. In some examples, the polyethylene comprises ultra-high-molecular-weight polyethylene (UHMWPE). In some examples, the polyethylene is ultra-high-molecular-weight polyethylene (UHMWPE). In some examples, the polyolefin comprises polypropylene. polypropylene PVC. In some examples, the multi-yarn thread 318 comprises at least one yarn comprising a biocompatible material. In some examples, the multi-yarn thread 318 comprises at least one yarn consisting of a biocompatible material(s).

In general and as disclosed herein, the multi-yarn thread 318 is thicker than the backing thread 320, at least in the free state of the multi-yarn thread. This may further be manifested in a distinction in the number of yarns of each of the multi-yarn thread 318 and the backing thread 320. Thus, according to some examples, the multi-yarn thread 318 comprises at least two yarns 370 and the backing thread 320 comprises a single yarn. In some examples, the multi-yarn thread 318 comprises at least four yarns 370 and the backing thread 320 comprises a single yarn. In some examples, the multi-yarn thread 318 comprises 4 to 16 yarns 370 and the backing thread 320 comprises a single yarn. In some examples, the multi-yarn thread 318 comprises a plurality of yarns 370 and the backing thread 320 comprises at least one yarn, wherein the number of the plurality of yarns 370 is at least two times greater than number of the yarn(s) of the backing thread 320. In some examples, the multi-yarn thread 318 comprises a plurality of yarns 370 and the backing thread 320 comprises at least one yarn, wherein the number of the plurality of yarns 370 is at least four times greater than number of the yarn(s) of the backing thread 320.

In some examples, each of the plurality of yarns 370 of the multi-yarn thread 318 is a bulk textured yarn which has a weight between 10 D to 150 D. In some examples, each of the plurality of yarns 370 of the multi-yarn thread 318 is a bulk textured yarn which has a weight between 12 D to 140 D. In some examples, each of the plurality of yarns 370 of the multi-yarn thread 318 is a bulk textured yarn which has a weight between 14 D to 130 D. In some examples, each of the plurality of yarns 370 of the multi-yarn thread 318 is a bulk textured yarn which has a weight between 15 D to 120 D. In some examples, each of the plurality of yarns 370 of the multi-yarn thread 318 is a bulk textured yarn which has a weight between 16 D to 110 D. In some examples, each of the plurality of yarns 370 of the multi-yarn thread 318 is a bulk textured yarn which has a weight between 17 D to 100 D. In some examples, each of the plurality of yarns 370 of the multi-yarn thread 318 is a bulk textured yarn which has a weight between 18 D to 90 D. In some examples, each of the plurality of yarns 370 of the multi-yarn thread 318 is a bulk textured yarn which has a weight between 20 D to 80 D.

In some examples, the multi-yarn thread 318 has a filament count of about 5 to 150 per yarn. In some examples, the multi-yarn thread 318 has a filament count of about 10 to about 48 per yarn. In some examples, the multi-yarn thread 318 has a filament count of at least 5 In some examples, the multi-yarn thread 318 has a filament count of at least 6 filaments per yarn. In some examples, the multi-yarn thread 318 has a filament count of at least 7 filaments per yarn. In some examples, the multi-yarn thread 318 has a filament count of at least 8 filaments per yarn. In some examples, the multi-yarn thread 318 has a filament count of at least 9 filaments per yarn. In some examples, the multi-yarn thread 318 has a filament count of at least 10 filaments per yarn.

In some examples, the multi-yarn thread 318 has a density between 5 to 50 PPI. In some examples, the multi-yarn thread 318 has a density between 10 to 20 PPI. In some examples, the multi-yarn thread 318 has a linear mass density, which is at least 10% greater than the linear mass density of the backing thread 320. In some examples, the multi-yarn thread 318 has a linear mass density, which is at least 50% greater than the linear mass density of the backing thread 320.

According to some examples, the present method further comprises a step (d) of providing a base layer 302 having a thickness T1 and characterized according to any exemplary base layer disclosed herein, and spreading the base layer 302 in the automated sewing machine 400. The base layer 302 can optionally be spread such that its inner surface 382 is facing the embroidery bobbin 420 and its outer surface 380 is facing the embroidery needle 410.

In some examples, T2 is at least 100% greater than T1. In some examples, T2 is at least 50% greater than T1. In some examples, T2 is at least 100% greater than T1. In some examples, T2 is at least 200% greater than T1. In some examples, T2 is at least 300% greater than T1. In some examples, T2 is at least 400% greater than T1. In some examples, T2 is at least 500% greater than T1.

According to some examples, the present method further comprises attaching the base layer 302 to the tensioning fixture 430. According to some examples, attaching the base layer 302 to the tensioning fixture 430 entails placing the base layer 302 at a spread state between the first portion 440a and the second fixture portion 440b and locking the first portion 440a and the second fixture portion 440b over the base layer 302.

In some examples, the tensioning fixture 430 is configured to control the tension of the base layer 302 in at least one direction. According to some examples, the present method further comprises positioning the tensioning fixture 430 with the base layer 302 attached thereto between the embroidery needle 410 and the embroidery bobbin 420. Such positioning can be performed such that the outer surface 380 of the base layer 302 is facing the embroidery needle 410 and the inner surface 382 is facing the bobbin 420.

As mentioned above, the base layer can comprise, in some examples, a fabric, which can be provided as a woven fabric formed with warp fibers 304 and weft fibers 306. In some examples, the base layer 302 comprises a tear-resistant fabric. In some examples, the base layer 302 is made of a tear-resistant fabric.

FIGS. 8A-8B show an initial position of the embroidery needle 410 and embroidery bobbin 420 prior to forming a stitch. In this position, the backing thread 320 is pulled from the bobbin in the vicinity of the impending stitch point 350. Thus, according to some examples, the present method further includes a step (e) of pulling a section of the backing thread 320 from the embroidery bobbin 420 toward the inner surface 382 of the base layer 302 at a first stitch point 350.

FIG. 9A illustrates penetration of the embroidery needle 410 through the base layer 302 at stitch point 350. Thus, according to some examples, the present method further includes a step (f) of passing the embroidery needle 410 with the multi-yarn thread 318 through the base layer 302 at the first stitch point 350a. In some examples, step (f) of passing the embroidery needle 410 with the multi-yarn thread 318 through the base layer 302 entails disposing a portion of the multi-yarn thread 318 over the outer surface 380 of the base layer 302. Passing the embroidery needle 410 through the base layer 302 can optionally be performed by moving the embroidery needle 410 in a first direction 50, from the outer surface 380 toward and past the inner surface 382 of the base layer 302.

FIG. 9B shows the multi-yarn thread 318 interlaced with the backing thread 320, thereby initializing stitch formation. As shown in FIG. 9B, lacing may be promoted by rotation of the hook 422, such that the backing thread 320 is revolved around the multi-yarn thread 318 held by the embroidery needle 410. Thus, according to some examples, the present method further includes a step (g) of lacing the multi-yarn thread 318 around the backing thread 320. According to some examples, lacing the multi-yarn thread 318 around the backing thread 320 is performed by using the rotary hook 422. Steps (g) and (h) can together entail, in some examples, winding the multi-yarn thread 318 around the backing thread 320. In some examples, steps (g) and (h) together entail winding the backing thread 320 around the multi-yarn thread 318.

FIG. 9C shows the embroidery needle 410 withdrawn from the base layer 302, back to its position in step (a) above the outer surface 380 of the base layer 302. Thus, according to some examples, the present method further includes a step (h) of withdrawing the embroidery needle 410 from the base layer 302, thereby forming a stitch of the multi-yarn thread 318 and the backing thread 320 at the first stitch point 350a. In some examples, step (h) of withdrawing the needle entails aligning the multi-yarn thread 318 and the backing thread 320 along the base layer 302. Withdrawing the needle can optionally be performed by moving the embroidery needle 410 in a second direction 52, which is opposite to the first direction 50. In some examples, the stitch formed following steps (f) to (h) is a lock stitch.

FIG. 9D shows the base layer 302 repositioned relative to the needle 410 and bobbin 420 subsequent to stitch formation at stitch point 350a, so as to position a region of the base layer 302 at a designated subsequent stitch point 350b between the needle 410 and the bobbin 410. Thus, according to some examples, the present method further includes a step (i) of moving the base layer 302 to position a subsequent stitch point 350b between the needle 410 and the bobbin 420. This can optionally be performed by translating the base layer 302 relative to the needle 410 and bobbin 420, and/or translating the needle 410 and bobbin 420 relative to the base layer 302. In some examples, step (i) of moving the base layer 302 entails extending a portion of the backing thread 320 over the inner surface 382 of the base layer 302.

In some examples, the tensioning fixture 430 is movable. In some examples, the tensioning fixture 430 is automatically movable. In some examples, step (i) of moving or translating the base layer 302 entails moving the tensioning fixture 430 in a desired direction, optionally controlled by an actuation mechanism associated with the tensioning fixture 430.

In some examples, the distance between two successive stitch points 350, such as first stitch point 350a and subsequent stitch point 350b, is in the range of 2 to 7 millimeters, including each value and sub-range within the specified range. In some examples, the tensioning fixture 430 is movable at a predetermined interval. In some examples, the tensioning fixture 430 is automatically movable a predetermined interval. In some examples, step (i) of moving or translating the base layer 302 entails moving the tensioning fixture 430 in a desired direction along a predetermined interval, optionally controlled by an actuation mechanism associated therewith. In some examples, the predetermined interval is in the range of 2 millimeters to 7 millimeters.

In some examples, the distance between two successive stitch points 350 is less than a predetermined upper threshold length value. As mentioned above, an upper threshold length value can be determined to prevent the formed outer portions 322 from being long enough to introduce risks of thread entanglement. In some examples, the distance between two successive stitch points 350 is greater than a predetermined lower threshold length value. A lower threshold length value can be determined as a minimal desired length that will allow the outer portion 322 to expand to a desired thickness between the successive stitch points 350, such as the free-state thickness T2.

According to some examples, the present method further comprises a step (j) of repeating steps (e) to (h) to form an additional stitch at the subsequent stitch point 350b. This results in the formation of an outer portion 322 of the multi-yarn thread 318 extending between the first stitch point 350a and the subsequent stitch point 350b. While the outer portion 322 of multi-yarn thread 318 extend over the outer surface 380 of base layer 302, the backing thread 320 similarly extends between the same stitch points 350, over the inner surface 382 of the base layer 302, optionally in parallel to the outer portion 322.

It is to be understood that repeating steps (e) to (h) as mentioned in step (j) refers to repeating these steps with respect to stitch point 350b instead of first stitch point 350a as literally phrased and described above with respect to these steps.

The multi-yarn thread 318 extends along outer portion 322 between successive stitch points 350 at a first tension, while the backing thread 320 extends between successive stitch points 350 at a second tension. When the outer skirt 300 is assembled on the frame 104 of prosthetic valve 100, the portion of the backing threads 320 extending between stitch points 350 are sandwiched between the base layer 302 and the frame 104. Thus, it may be desirable to keep the backing thread 320 relatively taut between successive stitch points 350. In contrast, it may be desired to avoid tensioning of the multi-yarn thread 318 between successive stitch points 350, so as to form a sufficient slack of material that can be relatively loose, allowing it to assume a free state of the multi-yarn thread 318 along at least a portion of the outer portion 322. Thus, in some examples, the second tension of the backing thread 320 between successive stitch points 350 is greater than the first tension of the multi-yarn thread 318 along the outer portion 322 defined between the same stitch points 350.

In some examples, the second tension of the backing thread 320 between successive stitch points 350 is at least 10% greater than the first tension of the multi-yarn thread 318 between the same successive stitch points 350. In some examples, the second tension is at least 20% greater than the first tension. In some examples, the second tension is at least 30% greater than the first tension. In some examples, the second tension is at least 40% greater than the first tension. In some examples, the second tension is at least 50% greater than the first tension. In some examples, the second tension is at least 60% greater than the first tension. In some examples, the second tension is at least 70% greater than the first tension. In some examples, the second tension is at least 80% greater than the first tension. In some examples, the second tension is at least 90% greater than the first tension. In some examples, the second tension is at least 100% greater than the first tension.

After forming a first outer portions 322 between two stitch points 350a, 350b according to steps (e) to (j), the method can further comprise an optional step (k) of repeating step (i), that is, moving the base layer 302 to a position of another subsequent stitch point 350 between the needle 410 and the bobbin 420, and then repeating steps (e) to (h) as described above to form another stitch at the subsequent stitch point 350, thereby forming at least one additional successive outer portion 322. It is to be understood that repeating steps (e) to (h) as mentioned in step (k) refers to repeating these steps with respect to the subsequent stitch point 350 instead of first stitch point 350a as literally phrased and described above with respect to these steps.

A plurality of outer portions 322 between a plurality of corresponding stitch points 350 can be formed by repeating step (k) as many times as required, to form a desired pattern of the multi-yarn thread 318 over base layer 302.

When a desired pattern is finally achieved, the outer skirt 300 can optionally be removed from the automated sewing machine 400, and the method can optionally further comprise a step (l) of attaching the outer skirt 300 to the prosthetic valve 100. This can optionally be achieved by attaching, such as by sutures or other couplers, the base layer 302, to the frame 104 or other components (such as an inner skirt or the leaflets 112) of the prosthetic valve 100, such that the inner surface 382 is facing the frame 104, and the outer portions 322 are facing radially outward, away from the frame, such as toward the surrounding anatomy (e.g., native annulus) at the site of implantation. As mentioned, the outer skirt 300 can optionally be attached to the mechanically actuated valve 100 illustrated in FIGS. 1A-1C, as well as to other types of mechanical valves, balloon-expandable valves, and self-expandable valves.

Releasing the outer skirt 300 prior to attaching it to the frame 104 can optionally include releasing the base layer 302 from the tensioning fixture 300. In some examples, the base layer 302 can optionally be cut to a desired shape, including a desired length, shape and orientation of any of the inflow edge portion 308, outflow edge portion 310, and first 312 and second 314 side edge portions, prior to step (d) of providing the base skirt 302 and spreading it. In some examples, the base layer 302 can optionally be provided in step (d) prior to being cut to the final desired shape of the skirt around the valve, and the method can optionally further comprise, subsequent to releasing the outer skirt 300 from the embroidery machine 400 and/or tensioning fixture 430 and prior to step (l) of attaching the skirt 300 to the prosthetic valve 100, cutting it to a desired shape, including a desired length, shape and orientation of any of the inflow edge portion 308, outflow edge portion 310, and first 312 and second 314 side edge portions.

In some examples, attaching the skirt 300, such as its base layer 302, to the frame 104, comprises wrapping the base layer 104 around the frame 104, and attaching the first 312 and second 314 side edge portions to each other to form an annular configuration of the base layer 302. Attached the side edge portions 312 and 314 to each other can optionally be performed by suturing, either through pre-formed apertures 316 or without pre-formed apertures, such as directly into the thickness of the overlapping sections along the side edge portions 312, 314.

In some examples, attaching the skirt 300, such as its base layer 302, to the frame 104, comprises suturing the inflow edge portion 308 and/or outflow edge portion 310 to struts of the frame 104.

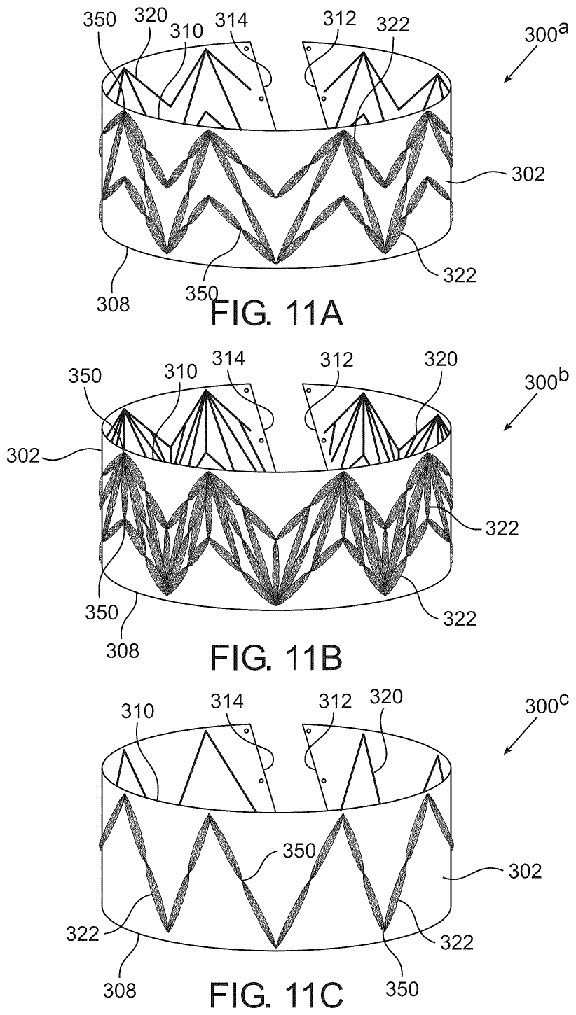

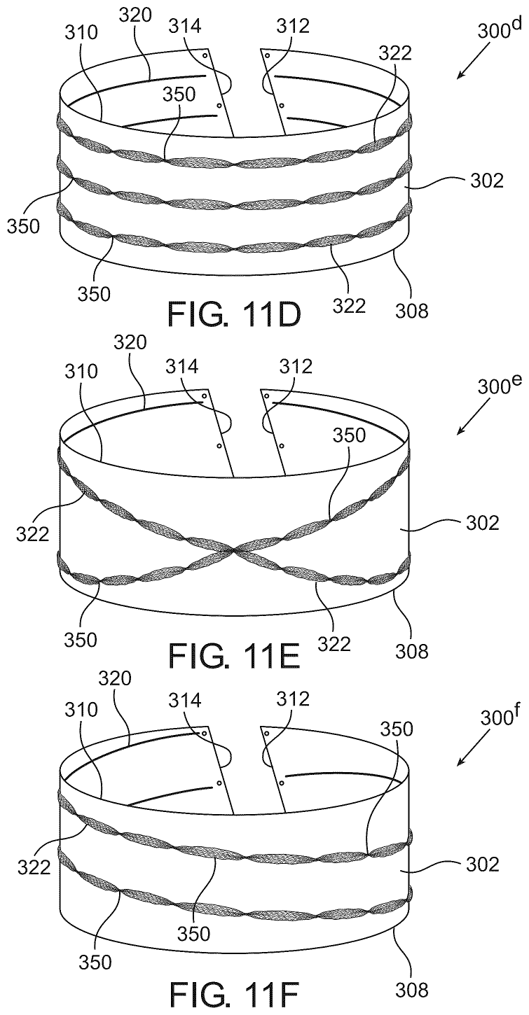

The above-mentioned method of embroidering the multi-yarn thread 318 to the base layer 302 can optionally be utilized to form any of the patterns described above with respect to FIGS. 3A-7D, as well as any other pattern of desire. FIGS. 11A-11F show patterned outer skirts 300 similar to those described with respect to FIGS. 3A-7D, formed according to the proposed embroidery patterning method.

FIG. 11A shows an outer skirt 300^{a} that includes a pattern similar to that described above with respect to FIGS. 3A-4, only that when formed according to the proposed embroidery patterning method, the skirt 300^{a} further comprises backing thread(s) 320 extending along the inner surface 382 of base layer 302, following the same pattern as that of the outer portions 322.

FIG. 11B shows an outer skirt 300^{b} that includes a pattern similar to that described above with respect to FIGS. 5A-6, only that when formed according to the proposed embroidery patterning method, the skirt 300^{b} further comprises backing thread(s) 320 extending along the inner surface 382 of base layer 302, following the same pattern as that of the outer portions 322.

FIG. 11C shows an outer skirt 300^{c} that includes a pattern similar to that described above with respect to FIG. 7A, only that when formed according to the proposed embroidery patterning method, the skirt 300^{c} further comprises backing thread(s) 320 extending along the inner surface 382 of base layer 302, following the same pattern as that of the first diagonal portions 324.

FIG. 11D shows an outer skirt 300^{d} that includes a pattern similar to that described above with respect to FIG. 7B, only that when formed according to the proposed embroidery patterning method, the skirt 300^{d} further comprises backing thread(s) 320 extending along the inner surface 382 of base layer 302, following the same pattern as that of the circumferential portions 338.

FIG. 11E shows an outer skirt 300^{e} that includes a pattern similar to that described above with respect to FIG. 7C, only that when formed according to the proposed embroidery patterning method, the skirt 300^{e} further comprises backing thread(s) 320 extending along the inner surface 382 of base layer 302, following the same pattern as that of the crossing angled circumferential portions 340.

FIG. 11F shows an outer skirt 300^{f} that includes a pattern similar to that described above with respect to FIG. 7D, only that when formed according to the proposed embroidery patterning method, the skirt 300^{f} further comprises backing thread(s) 320 extending along the inner surface 382 of base layer 302, following the same pattern as that of the parallel angled circumferential portions 340.

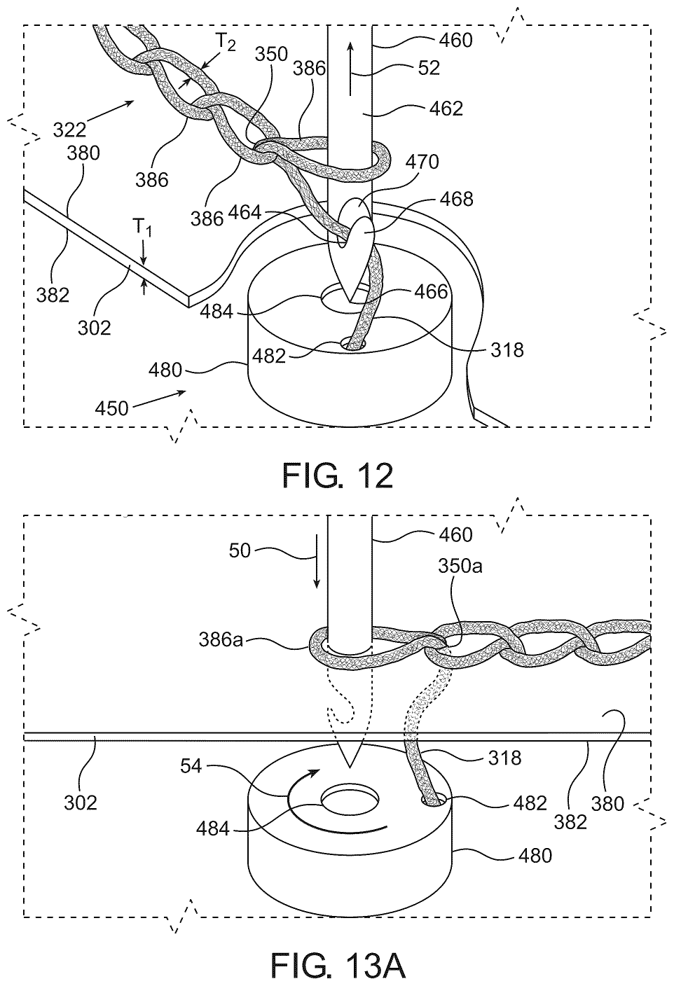

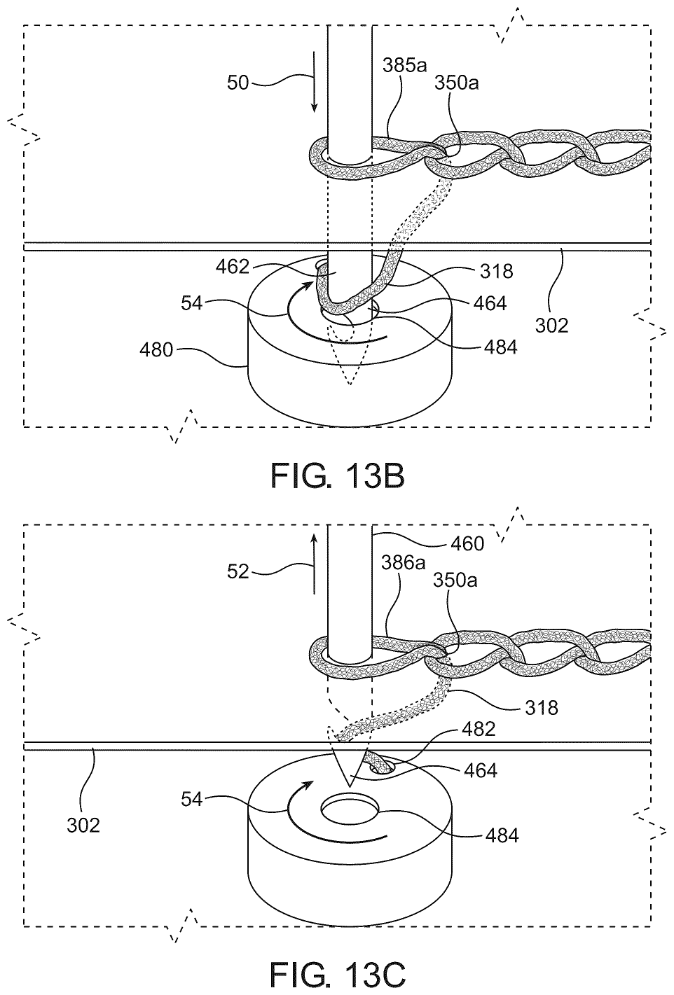

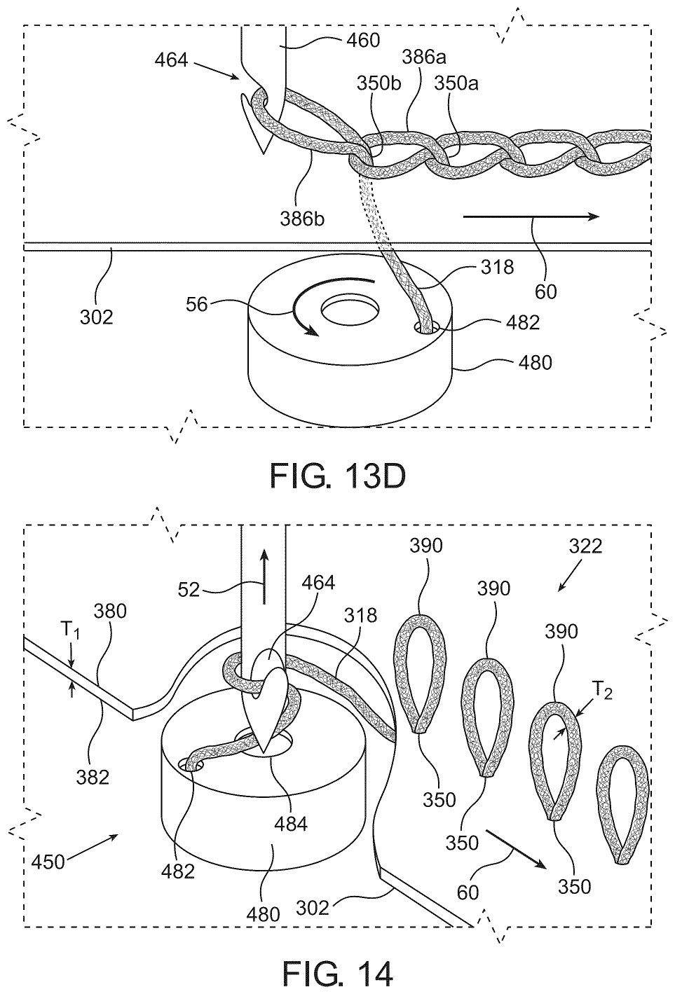

FIGS. 12-13D illustrate an exemplary chain-stitch embroidery patterning method for forming the outer skirt 300. In some examples, the chain-stitch embroidery patterning method disclosed herein is utilized for attaching the multi-yarn thread(s) 318 to the base layer 302, forming outer portions 322 of the multi-yarn thread 318 extending over the outer surface 380 of the base layer 304. Any one of the patterns described above with respect to FIGS. 3A-7D can optionally be formed by the method disclosed herein.

FIG. 12 shows a chain-stitch formed at a stitch point 350 upon pulling a portion of a multi-filament thread 318 carried by a bearded needle 460, through a previously formed loop, according to some examples. A portion of the base layer 302 is removed from view to expose components of an automated sewing machine 450 used in the procedure. FIGS. 13A-13D illustrate some steps of a chain-stitch embroidery patterning method.

In some examples, a chain-stitch embroidery patterning method comprises a step of providing an automated sewing machine 450. In some examples, the sewing machine 450 comprises a bearded needle 460 and a looper 480. A bearded needle 460, also known as a spiral-eye needle, comprises a shank 462 and a hooking portion 464 extending therefrom. The hooking portion 464 extends from the sharp tip 466 of the needle 460, and includes a hook 468 extending from the tip 466 and defining a needle eye 470 into which a suture, such as multi-yarn suture 318, can be inserted and carried by the needle.

The chain-stitch embroidery patterning method can optionally further comprise spreading a base layer 302 in the automated sewing machine 450 such that its inner surface 382 is facing the looper 480 and its outer surface 380 is facing the bearded needle 410. According to some examples, the sewing machine 450 further comprises tensioning fixture 430. In some examples, tensioning fixture 430, which is not necessarily part of the sewing machine 450, is separately provided to allow spreading and tensioning of the base layer 302. Tensioning fixture 430 can optionally be utilized to affix and/or tension the base layer 302 between the bearded needle 460 and the looper 480 in a similar manner to that described above with respect to automated sewing machine 400.

The looper 480 comprises a needle insertion hole 484 which is aligned with the bearded needle 460 and sized to allow passing of the needle 460 therethrough. The looper 480 further comprises a draw hole 482 through which the multi-yarn thread 318 is supplied. The draw hole 482 is offset from the needle insertion hole 484. The looper 480 is rotatable, such that rotation of the looper 480 can move the angular position of the draw hole 482 relative to the central axis passing through the needle insertion hole.

An outer portion 322, including any of diagonal portions 324, 326, 328, 330, 332, vertical portions 334, 336, and/or circumferential portions 338, 340, when formed following a chain-stitch embroidery patterning method, may be different than outer portions formed by lock-stitch embroidery patterning method in that an outer portion 322 of a chain-stitch embroidery patterning method can include a series of interlaced loops 386 disposed over the outer surface 380 of base layer 302. Moreover, the multi-yarn thread 318 is stitched to the base layer 302 without the use of a backing thread 320.

An initial loop 386a can be formed by hooking the multi-yarn thread 318 on the hooking portion 464 of bearded needle 460, and drawing the thread 318 from the draw hole 480 of looper 480 during elevation of the needle 460 in the second direction 52, i.e. away from the looper 480. The looper 460 can be rotated while the needle 460 is elevated to a top dead center, after which the rotation of looper 480 can be stopped, and the needle 460 is lowered in direction 50 to pierce through the base layer 302, as shown in FIG. 13A. As the needle 460 is lowered in direction 50 to pierce through base layer 302, the thread 318 of the previously formed loop 386a is off-hooked, i.e., released from the needle eye 470.

As further shown in FIG. 13B, the needle is further lowered in the first direction 52 and is inserted into needle insertion hole 484, such that the hooking portion is below the upper surface of looper 480. The looper 480 is rotated in a first rotational direction 54, shown as a clockwise direction in FIG. 13B, such that the multi-yarn thread 318 is wound around the shank 462.

FIG. 13C illustrates a subsequent step of elevating the needle in direction 52, while the looper 480 keeps rotating. The multi-yarn thread 318 is hooked by the hooking portion 464 during elevation of the needle 460 such that a new subsequent loop 386b is formed by further elevating the needle 460 through the previously formed loop 386a to the top dead center. As shown in FIG. 13D, the multi-yarn thread 318, hooked by the bearded needle 460, is passed through the previously formed loop 386a by passing the needle 460 through the previously formed loop 386a while rotating the looper 480 in a second rotational direction 56 (opposite to the first rotational direction 54, such as counterclockwise) to an initial angular position of the draw hole 482, and the base layer can then be moved, such as in direction 60 illustrated in FIG. 13D, to position a region at which a subsequent stitch point 350 is to be formed, between the needle 460 and the looper 480.

In this manner, a series of loops 386 can optionally be formed, interlaced with each other, together forming, as mentioned above, any of the outer portions 322 that can form a desired pattern as disclosed herein. For example, a first diagonal portion 324 can be formed by a series of interlaced loops 386 extending over the outer surface 380 of base layer 302, between an inflow stitch point 352 and an outflow stitch points 354. Since such a first diagonal portion 324 can optionally be formed by a plurality of loops 386, each extending from a corresponding stitch point 350, an outer portion 322 such as first diagonal portion 324, will include additional stitch points 350 disposed between the inflow stitch point 352 and the outflow stitch point 354, wherein the inflow 352 and outflow 354 stitch points serve as stitch points that define vertices or end portions of the outer portion 322 as a whole.

The length of each loop 386, and the distance between stitch points 350 of successive loops 386, can be chosen as desired by controlling the movement of needle 460 in direction 50, 52, the rotational movement of the looper 480 in directions 54, 56, and the movement of base layer 302 in direction 60.

When patterning of skirt 300 is complete, the skirt 300 can optionally be removed from the sewing machine 450 and/or tensioning fixture 430, and coupled to the frame 104 of prosthetic valve 100 in the same manner described above with respect to the lock-stitch embroidery patterning method.

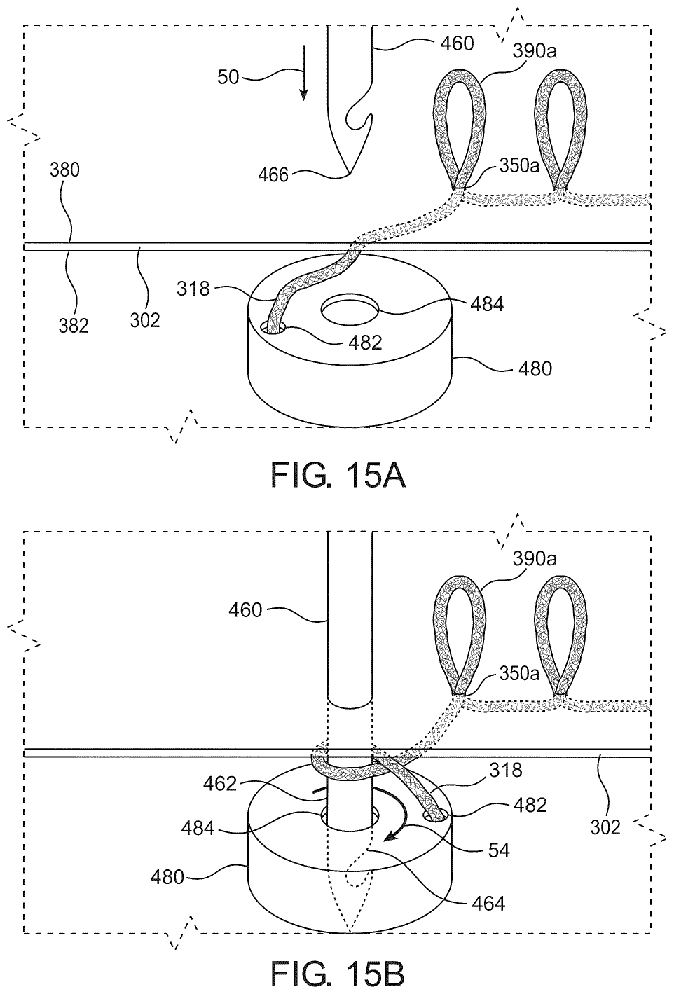

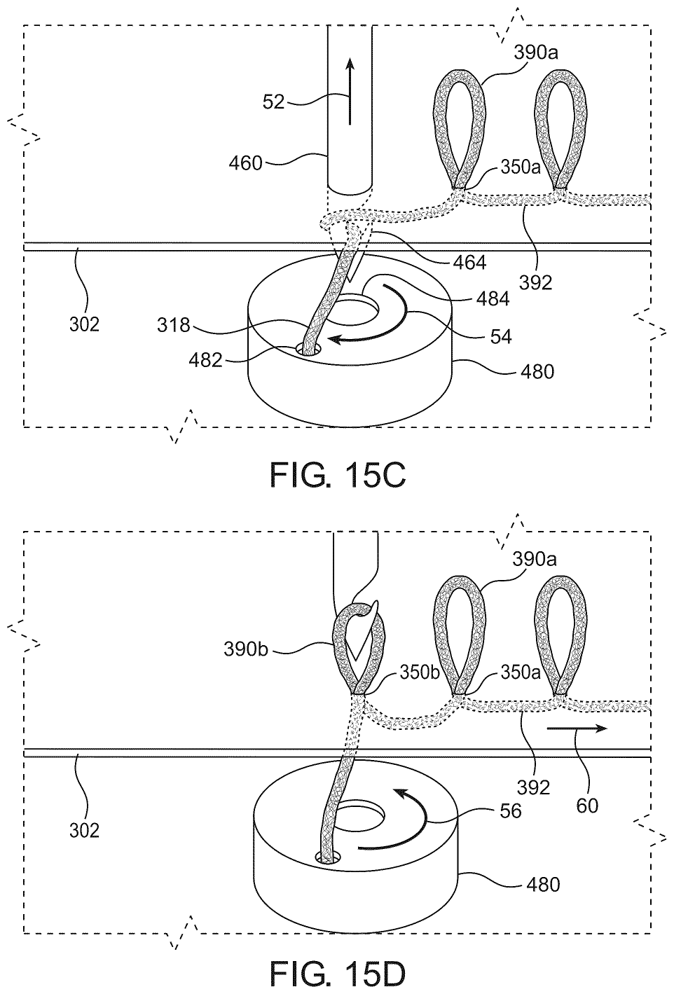

FIGS. 14-15D illustrate an exemplary moss-stitch embroidery patterning method for forming the outer skirt 300. In some examples, the moss-stitch embroidery patterning method disclosed herein is utilized for attaching the multi-yarn thread(s) 318 to the base layer 302, forming any desired patten of loops 390 extending radially away from the outer surface 380 of the base layer 304. In some examples, the moss-stitch embroidery patterning method disclosed herein is utilized for attaching the multi-yarn thread(s) 318 to the base layer 302, forming outer portions 322 of the multi-yarn thread 318 extending over the outer surface 380 of the base layer 304. Any one of the patterns described above with respect to FIGS. 3A-7D, as well as other patterns of desire, can optionally be formed by the method disclosed herein.

FIG. 14 shows a moss-stitch formed at a stitch point 350 upon pulling a portion of a multi-filament thread 318 carried by a bearded needle 460, through a previously formed loop, according to some examples. The same automated sewing machine 450 comprising the bearded needle 460 and the looper 480 described above for use in chain-stitch embroidery, can optionally be used for moss-stitch embroidery. A portion of the base layer 302 is removed from view to expose components of the automated sewing machine 450 used in the procedure. FIGS. 15A-15D illustrate some steps of a moss-stitch embroidery patterning method.

In some examples, a moss-stitch embroidery patterning method comprises a step of providing the automated sewing machine 450. The moss-stitch embroidery patterning method can optionally further comprise spreading a base layer 302 in the automated sewing machine 450 such that its inner surface 382 is facing the looper 480 and its outer surface 380 is facing the bearded needle 410. According to some examples, the sewing machine 450 further comprises tensioning fixture 430. In some examples, tensioning fixture 430, which is not necessarily part of the sewing machine 450, is separately provided to allow spreading and tensioning of the base layer 302. Tensioning fixture 430 can optionally be utilized to affix and/or tension the base layer 302 between the bearded needle 460 and the looper 480 in a similar manner to that described above.

An outer portion 322, including any of diagonal portions 324, 326, 328, 330, 332, vertical portions 334, 336, and/or circumferential portions 338, 340, when formed following a moss-stitch embroidery patterning method, may be different that outer portions formed by lock-stitch embroidery patterning method or chain-stitch embroidery patterning method in that an outer portion 322 of a moss-stitch embroidery patterning method can include a series of loops 390 extending away (e.g., radially outward when the resulting skirt 300 is coupled to a frame 104) from outer surface 380 of base layer 302, and spaced away from each other along the outer surface 380. At least some of the loops 390 can optionally be connected to each other by stitching intervals 392 of the multi-yarn thread 320 disposed over the inner surface 382 of the base layer 302. Thus, the multi-yarn thread 318 is stitched to the base layer 302 without the use of a backing thread 320, and the loops 390 are not interlaced with each other along the outer surface 380 of base layer 302.

FIG. 15A shows a state in which the bearded needle 460 is positioned at a top dead center, with at least one loop 390a formed over the base layer 302, extending away from the outer surface 380. In the state shown in FIG. 15A, the base layer 302 has been moved, such as in direction 60, relative to the previously formed loop 390a, to position a region of the base layer 302 through which a subsequent loop 350b is to be formed, aligned between the bearded needle 460 and the looper 480.

At this position, as shown in FIG. 15B, the needle 460 is lowered in direction 52 from the top dead center to pierce the base layer 302, and to be inserted into the needle insertion hole 484 of the looper 480, such that the hooking portion is below the upper surface of looper 480. The looper 480 is rotated in a first rotational direction 54, shown as a clockwise direction in FIG. 15B, such that the multi-yarn thread 318 extending from the draw hole 482 is wound around the shank 462.

FIG. 15C illustrates a subsequent step of elevating the needle in direction 52, while the looper 480 keeps rotating. The multi-yarn thread 318 is hooked by the hooking portion 464 during elevation of the needle 460, and as shown in FIG. 15D, a new subsequent loop 390b is formed by further elevating the needle 460 to the top dead center above the base layer 302. As the looper 480 is rotated in the second rotational direction 56 (opposite to the first rotational direction 54, such as counterclockwise), the loop 390b is off-hooked (i.e., released from hooking portion 464) by movement of the base layer in direction 60. A series of loops 390 can optionally be formed in sequence by repeating these steps.

During movement of the base layer from a previous stitch point, such as stitch point 350, to a subsequent stitch point, such as stitch point 350b, the multi-yarn thread 318 extends and can optionally be relatively tensioned between the successive stitch points 350, forming a stitching interval 392 of the multi-yarn thread 318 disposed over the inner surface 382 of the base layer 302 between successive stitch points 350. The length of the stitching intervals 392 along the inner surface 318 can be representative of the distance, along the outer surface 380, between the corresponding successive stitch points 350.

FIG. 16 shows a cross-sectional view of a portion of a skirt 300 along which a series of loops 390 are formed, spaced from each other along the outer surface 380 of the base layer 302, together optionally forming, as mentioned above, any of the outer portions 322 that can optionally form a desired pattern as disclosed herein. For example, a first diagonal portion 324 can optionally be formed by a series of extending outwardly from outer surface 380 of base layer 302, spaced from each other along the outer surface 380 between an inflow stitch point 352 and an outflow stitch points 354. Since such a first diagonal portion 324 can optionally be formed by a plurality of loops 390, each extending from a corresponding stitch point 350, an outer portion 322 such as first diagonal portion 324, will include additional stitch points 350 disposed between the inflow stitch point 352 and the outflow stitch point 354, wherein the inflow 352 and outflow 354 stitch points serve as stitch points that define vertices or end portions of the outer portion 322 as a whole.

The length of each loop 390, and the distance between successive loops 390 or length of stitching intervals 392, can be chosen as desired by controlling the movement of needle 460 in direction 50, 52, the rotational movement of the looper 480 in directions 54, 56, and the movement of base layer 302 in direction 60. Denser patterns, characterized by a greater number of loops 300 per unit of length, and in some cases, per unit of area, can be dictated by the length of stitching intervals 392 or distance between adjacent loops 390.

When patterning of skirt 300 is complete, the skirt 300 can optionally be removed from the sewing machine 450 and/or tensioning fixture 430, and coupled to the frame 104 of prosthetic valve 100 in the same manner described above with respect to the lock-stitch embroidery patterning method.

### ADDITIONAL EXAMPLES OF THE DISCLOSED TECHNOLOGY

Some examples of above-described implementations are enumerated below. It should be noted that one feature of an example in isolation or more than one feature of the example taken in combination and, optionally, in combination with one or more features of one or more examples below are examples also falling within the disclosure of this application.

Example 1. A prosthetic valve comprising: an annular frame movable between a radially compressed state and a radially expanded state; and an outer skirt disposed around the frame, wherein the outer skirt comprises: a base layer having an inner surface facing the frame and an outer surface facing away from the frame, a multi-yarn thread comprising a plurality of multi-filament yarns and defining a first thickness in a free state thereof; and a backing thread defining a second thickness; wherein the multi-yarn thread defines at least one outer portion extending over the outer surface of the base layer, between two stitch points extending through the base layer, at which the multi-yarn thread is wound around the backing thread; wherein the backing thread extends along the inner surface of the base layer, in parallel to the corresponding outer portion, between the corresponding two stitch points; and wherein the first thickness is greater than the second thickness.

Example 2. The prosthetic valve of any example herein, particularly example 1, wherein the frame comprises a plurality of intersecting struts, and wherein the base layer is sutured to one or more of the plurality of struts.

Example 3. The prosthetic valve of any example herein, particularly any one of examples 1 to 2, wherein the at least one outer portion is attached to the base layer by lock stitches formed at the stitch points.

Example 4. The prosthetic valve of any example herein, particularly any one of examples 1 to 3, wherein the distance between the two stitch points of the at least one outer portion is in the range of 2 millimeters to 7 millimeters.

Example 5. The prosthetic valve of any example herein, particularly any one of examples 1 to 4, wherein the at least one outer portion comprises a plurality of outer portions.

Example 6. The prosthetic valve of any example herein, particularly any one of examples 1 to 5, wherein the plurality of yarns are spaced from each other in the free state of the multi-yarn thread.

Example 7. The prosthetic valve of any example herein, particularly any one of examples 1 to 6, wherein the plurality of yarns extend in a wavy pattern along the multi-yarn thread in a free state of the multi-yarn thread.

Example 8. The prosthetic valve of any example herein, particularly any one of examples 1 to 7, wherein the multi-yarn thread has a minimal thickness at the stitch points, and gradually expands along portions thereof extending away from the stitch points.

Example 9. The prosthetic valve of any example herein, particularly example 8, wherein the first thickness is at least 100% greater than the minimal thickness of the multi-yarn thread at the stitch points.

Example 10. The prosthetic valve of any example herein, particularly example 9, wherein the first thickness is at least 200% greater than the minimal thickness of the multi-yarn thread at the stitch points.

Example 11. The prosthetic valve of any example herein, particularly any one of examples 8 to 10, wherein the multi-yarn thread assumes the first thickness at an intermediate point of the outer portion between the stitch points.

Example 12. The prosthetic valve of any example herein, particularly any one of examples 1 to 11, wherein the second thickness of the backing thread is uniform along its length.

Example 13. The prosthetic valve of any example herein, particularly any one of examples 1 to 12, wherein the first thickness is at least 200% greater than the second thickness.

Example 14. The prosthetic valve of any example herein, particularly any one of examples 1 to 13, wherein the first thickness is at least 500% greater than the second thickness.

Example 15. The prosthetic valve of any example herein, particularly any one of examples 1 to 14, wherein the multi-yarn thread is a braided thread.

Example 16. The prosthetic valve of any example herein, particularly any one of examples 1 to 15, wherein the multi-yarn thread comprises between 3 to 25 yarns.

Example 17. The prosthetic valve of any example herein, particularly any one of examples 1 to 16, wherein the multi-yarn thread comprises between 4 to 16 yarns.

Example 18. The prosthetic valve of any example herein, particularly any one of examples 1 to 17, wherein each of the plurality of yarns of the multi-yarn thread has a weight between 10 Denier to 150 Denier.

Example 19. The prosthetic valve of any example herein, particularly any one of examples 1 to 18, wherein each of the plurality of yarns of the multi-yarn thread has a weight between 20 Denier to 80 Denier.

Example 20. The prosthetic valve of any example herein, particularly any one of examples 1 to 19, wherein the multi-yarn thread has a filament count between 5 to 150 per yarn.

Example 21. The prosthetic valve of any example herein, particularly any one of examples 1 to 19, wherein the multi-yarn thread has a filament count between 10 to 48 per yarn.

Example 22. The prosthetic valve of any example herein, particularly any one of examples 1 to 21, wherein the multi-yarn thread has a density between 5 to 50 PPI.

Example 23. The prosthetic valve of any example herein, particularly any one of examples 1 to 21, wherein the multi-yarn thread has a density between 10 to 20 PPI.

Example 24. The prosthetic valve of any example herein, particularly any one of examples 1 to 23, wherein the multi-yarn thread has a linear mass density which is at least 10% greater than a linear mass density of the backing thread.

Example 25. The prosthetic valve of any example herein, particularly any one of examples 1 to 24, wherein the multi-yarn thread has a linear mass density which is at least 50% greater than a linear mass density of the backing thread.

Example 26. The prosthetic valve of any example herein, particularly any one of examples 1 to 25, wherein the multi-yarn thread comprises at least one yarn comprising a polyester or a copolymer thereof, thermoplastic polyurethane (TPU), a polyolefin or a combination thereof.

Example 27. The prosthetic valve of any example herein, particularly any one of examples 1 to 26, wherein the backing thread comprises between 1 to 3 yarns.

Example 28. The prosthetic valve of any example herein, particularly any one of examples 1 to 26, wherein the backing thread comprises a single yarn.

Example 29. The prosthetic valve of any example herein, particularly any one of examples 1 to 28, wherein the backing thread comprises a single flat yarn or a twisted yarn having between 2 to 16 twists per inch (TPI).

Example 30. The prosthetic valve of any example herein, particularly any one of examples 1 to 29, wherein the backing thread comprises at least one yarn, which has a weight between 7 Denier to 60 Denier.

Example 31. The prosthetic valve of any example herein, particularly any one of examples 1 to 29, wherein the backing thread comprises at least one yarn, which has a weight between 10 Denier to 40 Denier.

Example 32. The prosthetic valve of any example herein, particularly any one of examples 1 to 29, wherein the backing thread comprises at least one yarn, which comprises a polyester, ultra-high-molecular-weight polyethylene (UHMWPE), polytetrafluoroethylene (PTFE), silk, a thermoplastic polyurethane (TPU): UHMWPE hybrid or any combination thereof.

Example 33. The prosthetic valve of any example herein, particularly any one of examples 1 to 32, wherein the multi-yarn thread extends between the two stitch points at a first tension, and wherein the multi-yarn thread extends between the two stitch points at a second tension which is greater than the first tension.

Example 34. The prosthetic valve of any example herein, particularly example 33, wherein the second tension is at least 50% greater than the first tension.

Example 35. The prosthetic valve of any example herein, particularly example 33, wherein the second tension is at least 100% greater than the first tension.

Example 36. The prosthetic valve of any example herein, particularly any one of examples 1 to 35, wherein the outer skirt is prepared by an embroidery method comprising: (a) providing an automated sewing machine, which comprises an embroidery needle and an embroidery bobbin assembled to a rotary hook; (b) providing the backing thread, wherein the backing thread is coiled around the embroidery bobbin; (c) providing the multi-yarn thread, wherein the multi-yarn thread is held by the embroidery needle; (d) spreading the base layer in the automated sewing machine such that its inner surface is facing the embroidery bobbin and its outer surface is facing the embroidery needle; (e) pulling a section of the backing thread from the embroidery bobbin toward the inner surface of the base layer at a first stitch point of the two stitch points; (f) passing the needle with the multi-yarn thread through the base layer at the first stitch point; (g) lacing the multi-yarn thread around the backing thread using the rotary hook; (h) withdrawing the embroidery needle from the base layer, thereby forming a stitch of the multi-yarn thread and the backing thread at the first stitch point; (i) moving the base layer to position a subsequent stitch point of the two stitch points, between the embroidery needle and the embroidery bobbin; and (j) repeating steps (e) to (h) to form an additional stitch at the subsequent stitch point, thereby forming the outer portion of the multi-yarn thread extending between the first stitch point and the subsequent stitch point.

Example 37. The prosthetic valve of any example herein, particularly example 36, wherein the method of forming the outer skirt further comprises repeating, one or more times, step (i) to position another subsequent stitch point between the embroidery needle and the embroidery bobbing, and steps (e) to (h) to form another stitch at the subsequent stitch point, thereby forming at least one additional successive outer portion.

Example 38. The prosthetic valve of any example herein, particularly any one of examples 1 to 37, further comprising a valvular structure mounted within the frame and comprising a plurality of leaflets configured to regulate flow through the prosthetic valve.

Example 39. The prosthetic valve of any example herein, particularly example 38, wherein the plurality of leaflets comprises three leaflets.

Example 40. The prosthetic valve of any example herein, particularly any one of examples 1 to 39, further comprising an inner skirt disposed around an inner surface of the frame.

Example 41. The prosthetic valve of any example herein, particularly any one of examples 1 to 40, wherein the base layer comprises a fabric.

Example 42. The prosthetic valve of any example herein, particularly example 41, wherein the fabric comprises a woven fabric formed with warp fibers and weft fibers.

Example 43. The prosthetic valve of any example herein, particularly any one of examples 1 to 42, wherein the base layer comprises a tear-resistant fabric.

Example 44. An embroidery patterning method for forming and assembling an outer skirt of a prosthetic valve, the method comprising: (a) providing an automated sewing machine, which comprises an embroidery needle and an embroidery bobbin assembled to a rotary hook; (b) providing a backing thread, wherein the backing thread is coiled around the embroidery bobbin; (c) providing a multi-yarn thread, wherein the multi-yarn thread is held by the embroidery needle; (d) spreading a base layer in the automated sewing machine such that its inner surface is facing the embroidery bobbin and its outer surface is facing the embroidery needle; (e) pulling a section of the backing thread from the embroidery bobbin toward the inner surface of the base layer at a first stitch point; (f) passing the needle with the multi-yarn thread through the base layer at the first stitch point; (g) lacing the multi-yarn thread around the backing thread using the rotary hook; (h) withdrawing the embroidery needle from the base layer, thereby forming a stitch of the multi-yarn thread and the backing thread at the first stitch point; (i) moving the base layer to position a subsequent stitch point between the embroidery needle and the embroidery bobbin; (j) repeating steps (e) to (h) to form an additional stitch at the subsequent stitch point, thereby forming an outer portion of the multi-yarn thread extending between the first stitch point and the subsequent stitch point; and (l) attaching the base layer to a frame of a prosthetic valve, such that the inner surface of the base layer is facing the frame.

Example 45. The embroidery patterning method of any example herein, particularly example 44, further comprising, subsequent step (j) and prior to step (l), a step (k) of repeating, one or more times, step (i) to position another subsequent stitch point between the embroidery needle and the embroidery bobbing, and steps (e) to (h) to form another stitch at the subsequent stitch point, thereby forming at least one additional successive outer portion.

Example 46. The embroidery patterning method of any example herein, particularly any one of examples 44 to 45, wherein step (d) further comprises affixing the base layer, in a tensioned state thereof, in a tensioning fixture.

Example 47. The embroidery patterning method of any example herein, particularly example 46, further comprising, prior to step (l) of attaching the base layer to the frame, releasing the base layer from the tensioning fixture.

Example 48. The embroidery patterning method of any example herein, particularly any one of examples 44 to 46, further comprising, prior to step (l) of attaching the base layer to the frame, removing the base layer from the automated sewing machine.

Example 49. The embroidery patterning method of any example herein, particularly any one of examples 44 to 48, wherein step (l) further comprises wrapping the base layer around the frame.

Example 50. The embroidery patterning method of any example herein, particularly example 49, wherein step (l) further comprises suturing a first side edge portion of the base layer and a second side edge portion of the base layer to each other.

Example 51. The embroidery patterning method of any example herein, particularly any one of examples 44 to 50, wherein the stitch formed in step (h) is a lock stitch.

Example 52. The embroidery patterning method of any example herein, particularly any one of examples 44 to 51, wherein step (g) entails winding the multi-yarn thread around the backing thread.

Example 53. The embroidery patterning method of any example herein, particularly any one of examples 44 to 52, wherein the length of the outer portion is in the range of 2 millimeters to 7 millimeters.

Example 54. The embroidery patterning method of any example herein, particularly any one of examples 44 to 53, wherein the multi-yarn thread is a braided multi-yarn thread.

Example 55. The embroidery patterning method of any example herein, particularly any one of examples 44 to 54, wherein the multi-yarn thread comprises at least one yarn comprising a polyester or a copolymer thereof, thermoplastic polyurethane (TPU), a polyolefin or a combination thereof.

Example 56. The embroidery patterning method of any example herein, particularly any one of examples 44 to 55, wherein the multi-yarn thread comprises a plurality of multi-filament yarns.

Example 57. The embroidery patterning method of any example herein, particularly example 56, wherein the plurality of yarns are spaced from each other in the free state of the multi-yarn thread.

Example 58. The embroidery patterning method of any example herein, particularly example 57, wherein the plurality of yarns extend in a wavy pattern along the multi-yarn thread in a free state of the multi-yarn thread.

Example 59. The embroidery patterning method of any example herein, particularly any one of examples 56 to 58, wherein the multi-yarn thread comprises between 3 to 25 yarns.

Example 60. The embroidery patterning method of any example herein, particularly any one of examples 56 to 58, wherein the multi-yarn thread comprises between 4 to 16 yarns.

Example 61. The embroidery patterning method of any example herein, particularly any one of examples 56 to 60, wherein each of the plurality of yarns of the multi-yarn thread has a weight between 10 Denier to 150 Denier.

Example 62. The embroidery patterning method of any example herein, particularly any one of examples 56 to 60, wherein each of the plurality of yarns of the multi-yarn thread has a weight between 20 Denier to 80 Denier.

Example 63. The embroidery patterning method of any example herein, particularly any one of examples 56 to 62, wherein the multi-yarn thread has a filament count between 5 to 150 per yarn.

Example 64. The embroidery patterning method of any example herein, particularly any one of examples 56 to 62, wherein the multi-yarn thread has a filament count between 10 to 48 per yarn.

Example 65. The embroidery patterning method of any example herein, particularly any one of examples 56 to 64, wherein the multi-yarn thread has a density between 5 to 50 PPI.

Example 66. The embroidery patterning method of any example herein, particularly any one of examples 56 to 64, wherein the multi-yarn thread has a density between 10 to 20 PPI.

Example 67. The embroidery patterning method of any example herein, particularly any one of examples 56 to 66, wherein the multi-yarn thread has a linear mass density which is at least 10% greater than a linear mass density of the backing thread.

Example 68. The embroidery patterning method of any example herein, particularly any one of examples 56 to 66, wherein the multi-yarn thread has a linear mass density which is at least 50% greater than a linear mass density of the backing thread.

Example 69. The embroidery patterning method of any example herein, particularly any one of examples 56 to 68, wherein the backing thread comprises between 1 to 3 yarns.

Example 70. The embroidery patterning method of any example herein, particularly any one of examples 56 to 68, wherein the backing thread comprises a single yarn.

Example 71. The embroidery patterning method of any example herein, particularly any one of examples 56 to 70, wherein the backing thread comprises a single flat yarn or a twisted yarn having between 2 to 16 twists per inch (TPI).

Example 72. The embroidery patterning method of any example herein, particularly any one of examples 69 to 71, wherein the backing thread comprises at least one yarn, which has a weight between 7 Denier to 60 Denier.

Example 73. The embroidery patterning method of any example herein, particularly any one of examples 69 to 71, wherein the backing thread comprises at least one yarn, which has a weight between 10 Denier to 40 Denier.

Example 74. The embroidery patterning method of any example herein, particularly any one of examples 44 to 73, wherein the outer portion extends over the outer surface of the base layer, and wherein the backing thread extends along the inner surface of the base layer, in parallel to the corresponding outer portion.

Example 75. The embroidery patterning method of any example herein, particularly any one of examples 44 to 74, wherein the multi-yarn thread defines a first thickness in a free state thereof, and wherein the backing thread defines a second thickness which is less than the first thickness.

Example 76. The embroidery patterning method of any example herein, particularly example 75, wherein the multi-yarn thread assumes the first thickness at an intermediate point of the outer portion between the stitch points.

Example 77. The embroidery patterning method of any example herein, particularly any one of examples 75 to 76, wherein the second thickness of the backing thread is uniform along its length.

Example 78. The embroidery patterning method of any example herein, particularly any one of examples 75 to 77, wherein the first thickness is at least 200% greater than the second thickness.

Example 79. The embroidery patterning method of any example herein, particularly any one of examples 75 to 77, wherein the first thickness is at least 500% greater than the second thickness.

Example 80. The embroidery patterning method of any example herein, particularly any one of examples 44 to 75, wherein the prosthetic valve further comprises a valvular structure mounted within the frame and comprising a plurality of leaflets configured to regulate flow through the prosthetic valve.

Example 81. The embroidery patterning method of any example herein, particularly example 80, wherein the plurality of leaflets comprises three leaflets.

Example 82. The embroidery patterning method of any example herein, particularly any one of examples 44 to 81, wherein the prosthetic valve comprises an inner skirt disposed around an inner surface of the frame.

Example 83. The embroidery patterning method of any example herein, particularly any one of examples 44 to 82, wherein the base layer comprises a fabric.

Example 84. The embroidery patterning method of any example herein, particularly example 83, wherein the fabric comprises a woven fabric formed with warp fibers and weft fibers.

Example 85. The embroidery patterning method of any example herein, particularly any one of examples 44 to 84, wherein the base layer comprises a tear-resistant fabric.

Example 86. The embroidery patterning method of any example herein, particularly any one of examples 55 to 85, wherein the multi-yarn thread extends between the two stitch points at a first tension, and wherein the multi-yarn thread extends between the two stitch points at a second tension which is greater than the first tension.

Example 87. A prosthetic valve comprising: an annular frame movable between a radially compressed state and a radially expanded state; and an outer skirt disposed around the frame, wherein the outer skirt comprises: a base layer having an inner surface facing the frame and an outer surface facing away from the frame, and a multi-yarn thread comprising between 4 and 16 multi-filament yarns; wherein the multi-yarn thread defines at least one outer portion extending over the outer surface of the base layer, between two stitch points extending through the base layer, at which the multi-yarn thread is coupled to the base layer; wherein each of the yarns of the multi-yarn thread has a weight between 20 Denier to 80 Denier; and wherein the multi-yarn thread has a filament count between 10 to 48 per yarn.

Example 88. The prosthetic valve of any example herein, particularly example 87, wherein the multi-yarn thread is a braided multi-yarn thread.

Example 89. The prosthetic valve of any example herein, particularly any one of examples 87 to 88, wherein the multi-yarn thread has a density between 10 to 20 PPI.

Example 90. The prosthetic valve of any example herein, particularly any one of examples 87 to 89, wherein the multi-yarn thread comprises at least one yarn comprising a polyester or a copolymer thereof, thermoplastic polyurethane (TPU), a polyolefin or a combination thereof.

Example 91. The prosthetic valve of any example herein, particularly any one of examples 87 to 90, wherein the at least one outer portion is attached to the base layer by lock stitches formed at the stitch points.

Example 92. The prosthetic valve of any example herein, particularly any one of examples 87 to 91, wherein the distance between the two stitch points of the at least one outer portion is in the range of 2 millimeters to 7 millimeters.

Example 93. The prosthetic valve of any example herein, particularly any one of examples 87 to 92, wherein the at least one outer portion comprises a plurality of outer portions.

Example 94. The prosthetic valve of any example herein, particularly any one of examples 87 to 93, wherein the yarns are spaced from each other in the free state of the multi-yarn thread.

Example 95. The prosthetic valve of any example herein, particularly any one of examples 87 to 94, wherein the yarns extend in a wavy pattern along the multi-yarn thread in a free state of the multi-yarn thread.

Example 96. The prosthetic valve of any example herein, particularly any one of examples 87 to 95, wherein the multi-yarn thread has a minimal thickness at the stitch points, and gradually expands along portions thereof extending away from the stitch points.

Example 97. The prosthetic valve of any example herein, particularly any one of examples 87 to 96, wherein the at least one outer portion is not taut between the two stitch points.

Example 98. The prosthetic valve of any example herein, particularly any one of examples 87 to 97, further comprising a valvular structure mounted within the frame and comprising a plurality of leaflets configured to regulate flow through the prosthetic valve.

Example 99. The prosthetic valve of any example herein, particularly example 98, wherein the plurality of leaflets comprises three leaflets.

Example 100. The prosthetic valve of any example herein, particularly any one of examples 87 to 99, further comprising an inner skirt disposed around an inner surface of the frame.

Example 101. The prosthetic valve of any example herein, particularly any one of examples 87 to 100, wherein the base layer comprises a fabric.

Example 102. The prosthetic valve of any example herein, particularly example 101, wherein the fabric comprises a woven fabric formed with warp fibers and weft fibers.

Example 103. The prosthetic valve of any example herein, particularly any one of examples 87 to 102, wherein the base layer comprises a tear-resistant fabric.

Example 104. The prosthetic valve of any example herein, particularly any one of examples 87 to 103, wherein the at least one outer portion comprises a plurality of interlaced loops disposed over the outer surface of the base layer.

Example 105. The prosthetic valve of any example herein, particularly any one of examples 87 to 103, wherein the at least one outer portion comprises a plurality of loops extending radially outwards from the base layer, wherein the plurality of loops are spaced from each other along the outer surface of the base layer.

Example 106. The prosthetic valve of any example herein, particularly example 105, wherein the multi-yarn thread further defines a stitching interval disposed along the inner surface of the base layer, between each two successive loops of the plurality of loops. Example 107. A prosthetic valve comprising: an annular frame defining a longitudinal axis and movable between a radially compressed state and a radially expanded state; and an outer skirt disposed around the frame, wherein the outer skirt comprises: a base layer extending between an inflow edge portion and an outflow edge portion, the base layer having an inner surface facing the frame and an outer surface facing away from the frame, and one or more threads defining a plurality of outer portions, each outer portion extending over the outer surface of the base layer, between two stitch points of a plurality of stitch points at which the thread is coupled to the base layer; wherein the plurality of stitch points comprises: a plurality of inflow stitch points closer to the inflow edge portion relative to the rest of the plurality of stitch points; a plurality of outflow stitch points closer to the outflow edge portion relative to the rest of the plurality of stitch points; a plurality of first intermediate stitch points proximal to the inflow stitch points; and a plurality of second intermediate stitch points distal to the outflow stitch points; wherein the plurality of outer portions comprises: a plurality of first diagonal portions, each extending between one of the inflow stitch points and one of the outflow stitch points; a plurality of second diagonal portions, each extending between one of the inflow stitch points and one of the first intermediate stitch points; and a plurality of third diagonal portions, each extending between one of the outflow stitch points and one of the second intermediate stitch points; wherein the plurality of inflow stitch points and the plurality of outflow stitch points are circumferentially offset from each other; wherein the plurality of first intermediate stitch points are circumferentially aligned with the plurality of outflow stitch points; and wherein the plurality of second intermediate stitch points are circumferentially aligned with the plurality of inflow stitch points.

Example 108. The prosthetic valve of any example herein, particularly example 107, wherein the plurality of first diagonal portions are angled with respect to the longitudinal axis.

Example 109. The prosthetic valve of any example herein, particularly any one of examples 107 to 108, wherein the plurality of second diagonal portions are angled with respect to the longitudinal axis.

Example 110. The prosthetic valve of any example herein, particularly any one of examples 107 to 109, wherein the plurality of third diagonal portions are angled with respect to the longitudinal axis.

Example 111. The prosthetic valve of any example herein, particularly any one of examples 107 to 110, wherein each of the plurality of second diagonal portions is parallel to a corresponding one of the plurality of third diagonal portions, which is circumferentially aligned therewith.

Example 112. The prosthetic valve of any example herein, particularly any one of examples 107 to 111, wherein consecutive first diagonal portions together form a zig-zagged pattern around the circumference of the frame.

Example 113. The prosthetic valve of any example herein, particularly any one of examples 107 to 112, wherein consecutive second diagonal portions together form a zig-zagged pattern around the circumference of the frame.

Example 114. The prosthetic valve of any example herein, particularly any one of examples 107 to 113, wherein consecutive third diagonal portions together form a zig-zagged pattern around the circumference of the frame.

Example 115. The prosthetic valve of any example herein, particularly any one of examples 107 to 114, wherein two of the plurality of first diagonal portions and two of the plurality of second diagonal portions extend from each of the plurality of inflow stitch points.

Example 116. The prosthetic valve of any example herein, particularly any one of examples 107 to 115, wherein two of the plurality of first diagonal portions and two of the plurality of third diagonal portions extend from each of the plurality of outflow stitch points.

Example 117. The prosthetic valve of any example herein, particularly any one of examples 107 to 116, wherein the plurality of outer portions further comprises: a plurality of first vertical portions, each extending one of the inflow stitch points and one of the second intermediate stitch points; and a plurality of second vertical portions, each extending one of the outflow stitch points and one of the first intermediate stitch points.

Example 118. The prosthetic valve of any example herein, particularly example 117, wherein the plurality of first vertical portions and the plurality of second vertical portions are parallel with the longitudinal axis.

Example 119. The prosthetic valve of any example herein, particularly any one of examples 107 to 118, wherein the plurality of stitch points further comprises: a plurality of third intermediate stitch points defined on the second diagonal portions; a plurality of fourth intermediate stitch points defined on the third diagonal portions; and wherein the plurality of outer portions further comprises: a plurality of fourth diagonal portions, each extending one of the outflow stitch points and one of the third intermediate stitch points; and a plurality of fifth diagonal portions, each extending one of the inflow stitch points and one of the fourth intermediate stitch points.

Example 120. The prosthetic valve of any example herein, particularly example 119, wherein the plurality of fourth diagonal portions are angled with respect to the longitudinal axis.

Example 121. The prosthetic valve of any example herein, particularly any one of examples 119 to 120, wherein the plurality of fifth diagonal portions are angled with respect to the longitudinal axis.

Example 122. An embroidery patterning method for forming and assembling an outer skirt of a prosthetic valve, the method comprising: (a) providing an automated sewing machine, which comprises a bearded needle and a looper; (b) providing a multi-yarn thread extending through a draw hole of the looper; (c) spreading a base layer in the automated sewing machine such that its inner surface is facing the looper and its outer surface is facing the bearded needle; (d) piercing the base layer by the bearded needle by lowering the needle from a top dead center towards the looper; (e) winding the multi-yarn thread around a shank of the bearded needle by continued lowering of the needle so as to insert a hooking portion of the needle inside a needle insertion hole of the looper, while rotating the looper in a first rotational direction; (f) hooking the multi-yarn thread by the hooking portion while elevating the bearded needle from the needle insertion hole; (g) forming a loop extending from a stitch point by further elevating the bearded needle and the multi-yarn thread hooked thereon through the base layer and to the dead center; (h) stopping rotation movement of the looper in the first rotation direction; (i) rotating the looper in a second rotational direction opposite to the first rotational direction; (j) moving the base layer to position a subsequent stitch point between the bearded needle and the looper; (k) repeating steps (d) to (j) one or more times to form one or more additional loops at the subsequent one or more stitch points, thereby forming an outer portion of the multi-yarn thread comprising a plurality of loops; and (l) attaching the base layer to a frame of a prosthetic valve, such that the inner surface of the base layer is facing the frame.

Example 123. The embroidery patterning method of any example herein, particularly example 122, wherein step (g) further comprises passing the bearded needle and the multi-yarn thread hooked thereon through a previously formed loop, thereby interlacing the currently formed loop with the previously formed loop.

Example 124. The embroidery patterning method of any example herein, particularly example 122, wherein step (g) further comprises forming a stitching interval of the multi-yarn thread, extending over the inner surface of the base layer and extending from a previously formed stitch point to the currently formed stitch point.

Example 125. The embroidery patterning method of any example herein, particularly any one of examples 122 to 124, wherein step (c) further comprises affixing the base layer, in a tensioned state thereof, in a tensioning fixture.

Example 126. The embroidery patterning method of any example herein, particularly example 125, further comprising, prior to step (k) of attaching the base layer to the frame, releasing the base layer from the tensioning fixture.

Example 127. The embroidery patterning method of any example herein, particularly any one of examples 122 to 124, further comprising, prior to step (k) of attaching the base layer to the frame, removing the base layer from the automated sewing machine.

Example 128. The embroidery patterning method of any example herein, particularly any one of examples 122 to 127, wherein step (k) further comprises wrapping the base layer around the frame.

Example 129. The embroidery patterning method of any example herein, particularly example 128, wherein step (k) further comprises suturing a first side edge portion of the base layer and a second side edge portion of the base layer to each other.

Example 130. The embroidery patterning method of any example herein, particularly any one of examples 122 to 129, wherein the multi-yarn thread is a braided multi-yarn thread.

Example 131. The prosthetic valve of any example herein, particularly any one of examples 122 to 130, wherein the multi-yarn thread comprises at least one yarn comprising a polyester or a copolymer thereof, thermoplastic polyurethane (TPU), a polyolefin or any combination thereof.

Example 132. The embroidery patterning method of any example herein, particularly any one of examples 122 to 130, wherein the multi-yarn thread comprises a plurality of multi-filament yarns.

Example 133. The embroidery patterning method of any example herein, particularly example 132, wherein the plurality of yarns are spaced from each other in the free state of the multi-yarn thread.

Example 134. The embroidery patterning method of any example herein, particularly example 132, wherein the plurality of yarns extend in a wavy pattern along the multi-yarn thread in a free state of the multi-yarn thread.

Example 135. The embroidery patterning method of any example herein, particularly any one of examples 132 to 134, wherein the multi-yarn thread comprises between 3 to 25 yarns.

Example 136. The embroidery patterning method of any example herein, particularly any one of examples 132 to 134, wherein the multi-yarn thread comprises between 4 to 16 yarns.

Example 137. The embroidery patterning method of any example herein, particularly any one of examples 132 to 136, wherein each of the plurality of yarns of the multi-yarn thread has a weight between 10 Denier to 150 Denier.

Example 138. The embroidery patterning method of any example herein, particularly any one of examples 132 to 136, wherein each of the plurality of yarns of the multi-yarn thread has a weight between 20 Denier to 80 Denier.

Example 139. The embroidery patterning method of any example herein, particularly any one of examples 132 to 138, wherein the multi-yarn thread has a filament count between 5 to 150 per yarn.

Example 140. The embroidery patterning method of any example herein, particularly any one of examples 132 to 138, wherein the multi-yarn thread has a filament count between 10 to 48 per yarn.

Example 141. The embroidery patterning method of any example herein, particularly any one of examples 132 to 140, wherein the multi-yarn thread has a density between 5 to 50 PPI.

Example 142. The embroidery patterning method of any example herein, particularly any one of examples 132 to 141, wherein the multi-yarn thread has a density between 10 to 20 PPI.

Example 143. The embroidery patterning method of any example herein, particularly any one of examples 122 to 142, wherein the prosthetic valve further comprises a valvular structure mounted within the frame and comprising a plurality of leaflets configured to regulate flow through the prosthetic valve.

Example 144. The embroidery patterning method of any example herein, particularly example 143, wherein the plurality of leaflets comprises three leaflets.

Example 145. The embroidery patterning method of any example herein, particularly any one of examples 122 to 144, wherein the prosthetic valve comprises an inner skirt disposed around an inner surface of the frame.

Example 146. The embroidery patterning method of any example herein, particularly any one of examples 122 to 145, wherein the base layer comprises a fabric.

Example 147. The embroidery patterning method of any example herein, particularly example 146, wherein the fabric comprises a woven fabric formed with warp fibers and weft fibers.

Example 148. The embroidery patterning method of any example herein, particularly any one of examples 122 to 147, wherein the base layer comprises a tear-resistant fabric.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A prosthetic valve comprising:
an annular frame movable between a radially compressed state and a radially expanded state; and
an outer skirt disposed around the frame, wherein the outer skirt comprises:
a base layer having an inner surface facing the frame and an outer surface facing away from the frame,
a multi-yarn thread comprising a plurality of multi-filament yarns and defining a first thickness in a free state thereof; and
a backing thread defining a second thickness;
wherein the multi-yarn thread defines at least one outer portion extending over the outer surface of the base layer, between two stitch points extending through the base layer, at which the multi-yarn thread is wound around the backing thread;
wherein the backing thread extends along the inner surface of the base layer, in parallel to the corresponding outer portion, between the corresponding two stitch points; and
wherein the first thickness is greater than the second thickness.

2. The prosthetic valve of claim 1, wherein the at least one outer portion is attached to the base layer by lock stitches formed at the stitch points.

3. The prosthetic valve of claim 1, wherein the plurality of yarns extend in a wavy pattern along the multi-yarn thread in a free state of the multi-yarn thread.

4. The prosthetic valve of claim 1, wherein the multi-yarn thread has a minimal thickness at the stitch points, and gradually expands along portions thereof extending away from the stitch points.

5. The prosthetic valve of claim 1, wherein the second thickness of the backing thread is uniform along its length.

6. The prosthetic valve of claim 1, wherein the multi-yarn thread is a braided thread.

7. The prosthetic valve of claim 1, wherein each of the plurality of yarns of the multi-yarn thread has a weight between 10 Denier to 150 Denier.

8. The prosthetic valve of claim 1, wherein the multi-yarn thread has a filament count between 5 to 150 per yarn.

9. The prosthetic valve of claim 1, wherein the multi-yarn thread has a density between 5 to 50 PPI.

10. The prosthetic valve of claim 1, wherein the multi-yarn thread has a linear mass density which is at least 10% greater than a linear mass density of the backing thread.

11. The prosthetic valve of claim 1, wherein the backing thread comprises between 1 to 3 yarns.

12. The prosthetic valve of claim 1, wherein the backing thread comprises a single flat yarn or a twisted yarn having between 2 to 16 twists per inch (TPI).

13. The prosthetic valve of claim 1, wherein the backing thread comprises at least one yarn, which has a weight between 7 Denier to 60 Denier.

14. The prosthetic valve of claim 1, wherein the multi-yarn thread extends between the two stitch points at a first tension, and wherein the multi-yarn thread extends between the two stitch points at a second tension which is greater than the first tension.

15. An embroidery patterning method for forming and assembling an outer skirt of a prosthetic valve, the method comprising:
(a) providing an automated sewing machine, which comprises an embroidery needle and an embroidery bobbin assembled to a rotary hook;
(b) providing a backing thread, wherein the backing thread is coiled around the embroidery bobbin;
(c) providing a multi-yarn thread, wherein the multi-yarn thread is held by the embroidery needle;
(d) spreading a base layer in the automated sewing machine such that its inner surface is facing the embroidery bobbin and its outer surface is facing the embroidery needle;
(e) pulling a section of the backing thread from the embroidery bobbin toward the inner surface of the base layer at a first stitch point;
(f) passing the needle with the multi-yarn thread through the base layer at the first stitch point;
(g) lacing the multi-yarn thread around the backing thread using the rotary hook;
(h) withdrawing the embroidery needle from the base layer, thereby forming a stitch of the multi-yarn thread and the backing thread at the first stitch point;
(i) moving the base layer to position a subsequent stitch point between the embroidery needle and the embroidery bobbin;
(j) repeating steps (e) to (h) to form an additional stitch at the subsequent stitch point, thereby forming an outer portion of the multi-yarn thread extending between the first stitch point and the subsequent stitch point; and
(l) attaching the base layer to a frame of a prosthetic valve, such that the inner surface of the base layer is facing the frame.

16. The embroidery patterning method of claim 15, further comprising, subsequent step (j) and prior to step (l), a step (k) of repeating, one or more times, step (i) to position another subsequent stitch point between the embroidery needle and the embroidery bobbing, and steps (e) to (h) to form another stitch at the subsequent stitch point, thereby forming at least one additional successive outer portion.

17. The embroidery patterning method of claim 15, wherein the stitch formed in step (h) is a lock stitch.

18. The embroidery patterning method of claim 15, wherein step (g) entails winding the multi-yarn thread around the backing thread.

19. The embroidery patterning method of claim 15, wherein the multi-yarn thread is a braided multi-yarn thread.

20. A prosthetic valve comprising:
an annular frame defining a longitudinal axis and movable between a radially compressed state and a radially expanded state; and
an outer skirt disposed around the frame, wherein the outer skirt comprises:
a base layer extending between an inflow edge portion and an outflow edge portion, the base layer having an inner surface facing the frame and an outer surface facing away from the frame, and
one or more threads defining a plurality of outer portions, each outer portion extending over the outer surface of the base layer, between two stitch points of a plurality of stitch points at which the thread is coupled to the base layer;
wherein the plurality of stitch points comprises:
a plurality of inflow stitch points closer to the inflow edge portion relative to the rest of the plurality of stitch points;
a plurality of outflow stitch points closer to the outflow edge portion relative to the rest of the plurality of stitch points;
a plurality of first intermediate stitch points proximal to the inflow stitch points; and
a plurality of second intermediate stitch points distal to the outflow stitch points;
wherein the plurality of outer portions comprises:
a plurality of first diagonal portions, each extending between one of the inflow stitch points and one of the outflow stitch points;
a plurality of second diagonal portions, each extending between one of the inflow stitch points and one of the first intermediate stitch points; and
a plurality of third diagonal portions, each extending between one of the outflow stitch points and one of the second intermediate stitch points;
wherein the plurality of inflow stitch points and the plurality of outflow stitch points are circumferentially offset from each other;
wherein the plurality of first intermediate stitch points are circumferentially aligned with the plurality of outflow stitch points; and
wherein the plurality of second intermediate stitch points are circumferentially aligned with the plurality of inflow stitch points.

21. The prosthetic valve of claim 20, wherein each of the plurality of second diagonal portions is parallel to a corresponding one of the plurality of third diagonal portions, which is circumferentially aligned therewith.

22. The prosthetic valve of claim 20, wherein the plurality of outer portions further comprises:
a plurality of first vertical portions, each extending one of the inflow stitch points and one of the second intermediate stitch points; and
a plurality of second vertical portions, each extending one of the outflow stitch points and one of the first intermediate stitch points.

23. The prosthetic valve of claim 20, wherein the plurality of stitch points further comprises:
a plurality of third intermediate stitch points defined on the second diagonal portions;
a plurality of fourth intermediate stitch points defined on the third diagonal portions; and
wherein the plurality of outer portions further comprises:
a plurality of fourth diagonal portions, each extending one of the outflow stitch points and one of the third intermediate stitch points; and
a plurality of fifth diagonal portions, each extending one of the inflow stitch points and one of the fourth intermediate stitch points.
